(19) United States
(12) Patent Application Publication
(10) Pub. No.: US 2024/0240203 A1
(43) Pub. Date: Jul. 18, 2024

(54) ADENOVIRAL VECTORS AND VACCINES THEREOF

(71) Applicant: SpyBiotech Limited, Oxford (GB)

(72) Inventors: Matthew DICKS, Oxford (GB); Sumi BISWAS, Oxford (GB)

(21) Appl. No.: 18/558,898

(22) PCT Filed: May 4, 2022
(86) PCT No.: PCT/GB2022/051137

§ 371 Date: Nov. 3, 2023

(30) Foreign Application Priority Data
May 4, 2021 (GB) 2106361.5
Nov. 23, 2021 (GB) 2116831.5

(51) Int. Cl. C12N 15/86; A61K 39/00; A61K 39/215; C07K 14/005

(52) U.S. Cl. CPC C12N 15/86; A61K 39/215; C07K 14/005; A61K 2039/5254; A61K 2039/5256; C12N 2710/10034; C12N 2710/10043; C12N 2710/10051; C12N 2760/16022; C12N 2770/20022

(57) ABSTRACT

The present disclosure relates to adenoviral vector comprising a transgene encoding an antigen having a T cell epitope. The vector capsid comprising a modified capsid protein having a first peptide partner. A second peptide partner is attached to the first peptide partner to provide a covalently linked peptide binding pair. The second peptide partner also being attached to an antigen, the antigen having a B cell epitope. In a preferred embodiment the transgene encoding one antigen is in the lumen of the viral capsid and the second peptide attached to the second antigen is displayed on the surface of the viral capsid. Further aspects of the invention relate to vaccines comprising said vector, its use in therapy and methods of manufacture and treatment thereof.

### FIELD OF THE INVENTION

The invention relates to adenoviral vectored vaccines expressing a transgene and having modified capsid proteins decorated with an antigen wherein the antigen expressed by the transgene encodes at least one T cell epitope and the antigen decorating the capsid encodes at least one B cell epitope, to vaccines containing such vectors, and methods for using and making such vectors and vaccines.

### BACKGROUND TO THE INVENTION

Adenoviruses (Ads) have an icosahedral protein capsid that surrounds the linear duplex genome. No lipid envelope is present. The capsid includes the structural proteins hexon, fiber, penton, IIIa, VIII and IX. It is thought that the fiber capsid protein aids attachment to the host cell, via the knob domain. Ads rely upon host infection in order to be able to replicate using the host cell's replication machinery. There are at least 57 serotypes of human adenovirus, Ads1-57 that may be grouped into seven “species” A-G. Similarly, animal Ads exist, such as canine and equine Ads, also classifiable into various serotypes and “species”. Serotypes are generally defined by the ability of antisera to neutralise the infection of cells in vitro. These viruses are well studied and understood, they can be grown in high titres, and they can infect both dividing and non-dividing cells and can be maintained in host cells as an episome. These characteristics make them a good therapeutic choice, since nearly all trials have shown they are safe and well-tolerated.

The icosahedral capsid is made up of several proteins. Hexon is the major protein forming the 20 triangular faces of the viral capsid. The hexon proteins form trimers, and each trimer interacts with six other trimers. The 12 vertices are formed by the penton capsomere, these are a complex of 3 fiber proteins and five penton proteins. A long fiber extends from each vertex, composed of three identical chains that form a knob at the end. The capsid also includes minor proteins, notably amongst them pIIIa, pVI, pVIII and pIX. These minor capsid proteins may be located on the inner or outer surface of the capsid and may have additional functions beyond structural ones. Exemplarily, pVI may facilitate nuclear import of hexon proteins and pIX may be involved with DNA packaging into the capsid and transcriptional activation.

Ads are commonly used for gene therapy, in particular as gene delivery vectors, due to their capacity for inclusion of additional genetic sequences. Over 2,000 gene therapy trials have been conducted using Ads. Adenoviral vectors allow for the transmission of the transgene they carry into the host nucleus but do not integrate viral DNA into the host chromosome. The insert size for Ads when used as gene therapy vectors is large, with a capacity of 8-36 kb possible.

Additionally, Ads have emerged as a promising vaccine delivery vehicle due to their ability to induce both innate and adaptive immune responses; having the capacity to induce potent antigen-specific B and T cell immune responses. Adenoviral vectors are highly immunogenic and are efficient in delivering antigens.

Several adenoviral vector based vaccine candidates were developed and pursued further in clinical trials, however, many of these were not successful. Adenovirus of the human serotype 5 (Ad5) based vaccine, developed against HIV-1 by Merck, induced CD8+ T cell responses but failed to prevent HIV infections. More recently the utility of Adenoviral vectored vaccines have been demonstrated by the SARS-CoV-2 pandemic and the use of adenoviral vaccines from Johnson and Johnson and Astra Zeneca which both express the “S” protein from SARS-CoV-2 in adenoviral vectors.

Nonetheless, a major obstacle to the continued success of adenoviral based vectors in human and animal therapy is the neutralisation of the vector by adenovirus-specific antibodies. Natural infection by adenovirus is high in human and animal populations, and therefore the adaptive immune system may recognise and respond to the presence of adenoviral vectors by the secretion of neutralising antibody (NAB). Similarly, the innate immune system may also be responsible for assisting the response to adenoviral vectors. It is estimated that 50% to 90% of the adult population has pre-existing immunity to Ad5 for example.

Such a response can clear the therapeutic adenoviral vectors before the desired effect is seen. Solving this issue would enable adenoviral therapeutic vectors to be more routinely used.

In our earlier application PCT/GB2020/052774, published as WO2021/084282 (herein incorporated by reference) we have demonstrated that it possible to decorate the surface of the adenoviral with an antigen of interest which allows shielding the vector from anti-vector antibodies by using specific binding peptide pairs.

Use of peptide binding pairs, such as SpyCatcher and SpyTag (WO2011/098772), based upon attachment proteins from a bacterium, has been established as a technology to irreversibly conjugate recombinant proteins and the like. Bio-conjugation between entities that would be impossible to achieve through genetic fusion between proteins can work using peptide binding pairs. Various Catcher and Tag pairs are now available, some based upon modifications of SpyCatcher and SpyTag and others based upon similar chemistry from alternative bacterial proteins.

### SUMMARY OF THE INVENTION

The present invention provides further improvements to such vectors.

Advantageously, for the first time, the inventors demonstrate that the surface of the virus can be decorated with larger antigenic proteins that have an advantageous effect of blocking the neutralisation of the virus by potent neutralising monoclonal antibody whilst maintain viral infectivity and allowing for the expression of a transgene to produce robust B and T cell immunity against both the antigen encoded by the transgene and the antigen decorating the vector. Should the antigen decorating the vector be sufficiently large, the displayed antigens may also protect the viral vaccine vector from neutralisation by host antibodies. This ensures the viral vaccine vector can be used for multiple immunizations without reduction in efficacy.

According to a first aspect the invention provides an adenoviral vector encoding a heterologous transgene encoding an antigen and wherein the vector has at least one modified capsid protein, said modification comprises the inclusion of a first peptide partner covalently bonded to a second peptide partner, wherein the second partner is attached to an antigen, characterised in that the antigen encoded by the transgene has at least one T cell epitope and the antigen attached to the second partner has at least one B cell epitope.

In a second aspect the invention provides an adenoviral vector encoding a heterologous transgene encoding an antigen and wherein the vector has at least one modified capsid protein, said modification comprises the inclusion of a first peptide partner covalently bonded to a second peptide partner, wherein the second partner is attached to an antigen, characterised in that the antigen encoded by the transgene and the antigen attached to the second partner share at least one epitope.

In a third aspect of the invention there is provided an adenoviral vector encoding a heterologous transgene encoding an antigen and wherein the vector has at least one modified capsid protein, said modification comprises the inclusion of a first peptide partner covalently bonded to a second peptide partner, wherein the second partner is attached to an antigen, characterised in that the antigen encoded by the transgene and the antigen attached to the second partner are derived from the same pathogen.

In an embodiment the amino acid sequence of antigen encoded by the transgene and the amino acid sequence of the antigen attached to the second partner are 40, 50, 60, 70, 80, 90, 95, 97, 99, 100% identical. In an alternative embodiment the antigen encoded by the transgene and the antigen attached to the second partner are different antigens.

The vectors of the invention optimise the production of both humoral and cellular immunogenicity directed against the pathogen or target when given to a human or other mammal.

The modified capsid protein may be any capsid protein but is preferably a hexon protein or pIX protein.

Further, in an embodiment, the modified capsid protein is a hexon protein and modified in a HVR loop.

As used herein for any aspect of the invention, the first and second peptide partner is part of a pair of peptides that are capable of forming a covalent bond, such as an isopeptide bond or ester bond under the appropriate conditions. These are also known as protein tag and catcher pairs or protein tag and binding partner pairs. The first peptide partner may be a first peptide tag or may be a first peptide catcher. Each partner pair may comprise a tag and a catcher. The covalent bond that is formed may spontaneously react, or require the assistance of a third entity such as a ligase. Further information on suitable peptide pairs is included below.

The first peptide partner and the second peptide partner form a peptide partner pair, which may be covalently linked by an isopeptide or ester bond, preferably an isopeptide bond.

The partner pair can be described as a two-part linker comprising a first and second partner that are capable of spontaneously forming an isopeptide bond. In particular, the two-part linker may be viewed as a peptide tag and polypeptide binding partner cognate pair (or “catcher”) that can be conjugated via a covalent bond when contacted under conditions that allow the spontaneous formation of an isopeptide bond between the peptide tag and its polypeptide binding partner. Such are known to those skilled in the art.

The first and second peptide partners may be derived from fibronectin-binding proteins. SpyTag and its partner SpyCatcher were derived from Streptococcus pyogenes fibronectin-binding protein, FbaB. SdyTag and SdyCatcher were constructed based on the native Cna protein B-type (CnaB) domain from a related fibronectin-binding protein in Streptococcus dysgalactiae. Further derivatives of such peptide partners are also now available, such as QueenCatcher, Mooncake and Katl as modified catchers and RumTag, RumTrunkTag, BacTag and PhoTag as tags. The pair Isopeptag/Pilin-C was created from protein Spy0128 of Streptococcus pyogenes. SnoopTag/SnoopCatcher was developed from the RrgA protein of Streptococcus pneumoniae that has no cross-reactivity with SpyTag/SpyCatcher.

In one aspect, the first peptide partner is the “tag” partner, which may be covalently linked by an isopeptide or ester bond to a second peptide partner which is a “catcher”. In this aspect, the capsid protein is preferably a hexon protein. Thus, the first peptide partner or “tag” which modifies the hexon is preferably a DogTag, Isopeptag, Isopeptag-N, SdyTag, PsCsTag or Jo. It is preferred that the first peptide partner or tag is not SpyTag. In this embodiment, SpyTag is unmodified during its insertion into the hexon protein.

In an alternative aspect, the first peptide partner is the “catcher” partner, which may be covalently linked by an isopeptide or ester bond to a second peptide partner which is a “tag”. In this aspect, the capsid protein is preferably a hexon protein. Thus, the first peptide partner or “catcher” which modifies the hexon may be a DogCatcher, SpyCatcher, SnoopCatcher, Pilin-C, Pilin-N, SdyCatcher, PsCsCatcher or In.

In one aspect, the first peptide partner is the “catcher” partner, which may be covalently linked by an isopeptide or ester bond to a second peptide partner which is a “tag”. In this aspect, the capsid protein is preferably a pIX protein. Thus, the first peptide partner or “catcher” which modifies the pIX is preferably a SpyCatcher, DogCatcher, SnoopCatcher, Pilin-C, Pilin-N, SdyCatcher, PsCsCatcher or In.

In an alternative aspect, the first peptide partner is the “tag” partner, which may be covalently linked by an isopeptide or ester bond to a second peptide partner which is a “catcher”. In this aspect, the capsid protein is preferably a pIX protein. Thus, the first peptide partner or “tag” which modifies the pIX protein is preferably a SpyTag, Snooptag, SnoopTagJr, DogTag, Isopeptag, Isopeptag-N, SdyTag, PsCsTag or Jo.

In one aspect, the first peptide partner may be inserted into a hexon protein, and optionally the insertion into the hexon protein may be up to 200, up to 150 or up to 100 amino acids in length. The insertion into the hexon protein may be at any appropriate point, optionally in any one or more of the hypervariable (HVR) loops. The first peptide partner inserted into the hexon protein may be a

DogTag. DogTag is capable of forming a spontaneous covalent bond with DogCatcher, or a covalent bond with SnoopTagIr or SnoopTag in a reaction requiring a catalyst, SnoopLigase. DogCatcher or SnoopTagJr or SnoopTag may therefore be the second peptide partner. DogTag or SnoopTagJr may therefore be the second peptide partner. The second peptide partner is linked or attached to an antigen. It is surprising to the inventors that DogTag was able to be inserted into the hexon capsid protein to form a functional adenovirus vector for capsid display of protein partners after the failure of the SpyTag insertion, as described above.

According to one aspect of the invention there is provided a vector wherein the first and second peptide partners are selected from the group of first and second pairs:
SpyCatcher and SpyTag;
SnoopCatcher and SnoopTagJr
DogCatcher and DogTag
SnoopTagJr and SnoopCatcher
SpyTag and SpyCatcher
   provided that when the modified capsid protein is a hexon protein the first partner is not SpyCatcher.

Other first peptide partners that may be included within the hexon protein that are less than 100 amino acids in length include:
Isopeptag which pairs with Pilin-C
Isopeptag-N which pairs with Pilin-N
SdyTag which pairs with SdyCatcher
PsCsTag which pairs with PsCsCatcher
Jo which pairs with In,
DogTag which pairs with SnoopTag/SnoopTagJr using SnoopLigase or
RrgATag/RrgATag2/DogTag which pair with RrgACatcher (and DogCatcher derived therefrom: “DogCatcher”).

It is preferred that of the possible peptide partners that are included within the hexon protein, the inclusion is not an insertion of SpyTag.

The fibronectin binding protein, FbaB, from Streptococcus pyogenes contains a CnaB2 adhesin domain. CnaB2 is stabilized by a spontaneous reaction of Lys and Asp side chains to form an isopeptide bond. CnaB2 has been split into the 13-residue SpyTag peptide and the 116-residue SpyCatcher protein.

In another aspect, the first peptide partner may be fused to the pIX capsid protein, optionally at the N- or C-terminal end, preferably at the C-terminal end. The first peptide partner fused to the pIX capsid protein may be a SpyCatcher, SnoopCatcher or DogCatcher. SpyCatcher is capable of forming a covalent bond with SpyTag, which herein forms the second peptide partner, and can therefore be attached to an antigen. SnoopCatcher is capable of forming a covalent bond with either SnoopTag or SnoopTagJr, and DogCatcher is capable of forming a covalent bond with DogTag, and may be used as a binding pair in either orientation as first or second peptide partner. The first peptide partner may also be a DogTag, SpyTag, SnoopTagJr or SnoopTag, wherein the matching second peptide partner is DogCatcher, SnoopTagJr, SnoopTag, SpyCatcher, DogTag, or SnoopCatcher. Other peptide partner pairs that may be suitable for fusion with pIX in either orientation are: RrgATag/RrgATag2/DogTag and RrgACatcher, Isopeptag/Pilin-C, Isopeptag-N/Pilin-N, SdyTag/SdyCatcher, PsCsTag/PsCsCatcher and Jo/In.

Particularly preferred may be the insertion of SnoopCatcher or DogCatcher into pIX, since these have both been demonstrated herein to have good adenoviral viability. These insertions are furthermore genetically stable for greater than 3 passages.

Said adenoviral vector may be used in the preparation of a vaccine. The vaccine may be prophylactic or therapeutic. The invention therefore extends to a method of preparing a vaccine, and to vaccine formulations of an adenoviral vector as described herein.

Accordingly, in an aspect of the invention there is provided a method for manufacturing a vaccine formulation comprising admixing a vector of the invention with a pharmaceutically acceptable excipient.

The present invention further provides a method for the production of the vectors of the invention. The method comprises the
i. Introducing a nucleic acid which encodes a first peptide partner into the nucleic acid encoding a capsid protein of an adenovirus
ii. Introducing a transgene encoding an antigen into the adenoviral genome
iii. Infecting a cell with the adenovirus and collecting the progeny
iv. Attaching an second peptide partner attached to an antigen to the first peptide partner wherein the vector produced by the method of the invention is characterised by the antigen encoded by the transgene having at least one T cell epitope, and the antigen attached to the second peptide partner having at least one B cell epitope

The second peptide partner is attached to the antigen, preferably fused to said antigen, and is capable of forming a covalent bond with the first peptide partner present on the immunogenic adenoviral vector. The covalent bond, and therefore attachment, may occur spontaneously, or may require the use of a third entity to facilitate binding, such as a ligase. Thus, the antigen is attached to the adenovirus by means of the peptide partner pair, the first partner of which is included within a modified capsid protein.

In another aspect of the invention, there is provided a vaccine composition comprising an adenoviral vector as herein described in admixture with a pharmaceutically acceptable excipient.

In another aspect of the invention, there is provided an immunogenic adenoviral vector encoding a transgene having a T cell epitope, said vector comprising at least one modification in the hexon capsid protein, wherein said modification comprises: a first peptide partner; and a second peptide partner attached to an antigen having at least one B cell epitope, wherein the first peptide partner and second peptide partner are coupled via a covalent bond.

In an embodiment the antigen encoded by the transgene or the antigen attached to the second partner is an antigen selected from the group, viral, bacterial, parasitic, or fungal antigen. In an embodiment the antigen comprises the receptor binding domain from a pathogen.

In an embodiment the antigen attached to the second peptide partner is selected from the group: CSP protein from Plasmodium spp, particularly Plasmodium falciparum or Plasmodium Vivax, Human Cytomegalovirus (HCMV) gB; SARS CoV-2 S protein, Influenza heamagglutinin (HA) or fragments thereof containing at least one B cell epitope. Preferred fragments include NANP 18 from the malarial CSP protein; the receptor binding domain (RBD) domain from the SARS-CoV-2 S protein and the RBD binding domain from Influenza HA.

In general the receptor binding domain of any antigen is a preferred fragment to surface decorate the vector. That is to say RBDs are as a class a preferred antigen to attach to the second peptide partner.

In an embodiment the transgene encodes an antigen selected from the group: HCMV pentamer; SARS -CoV-2 S protein, Influenza nucleoprotein or fragments thereof encoding at least one T cell epitope.

### FIGURES

The disclosure will be more clearly understood by reference to the accompanying Figures, in which:

FIG. 1: shows a schematic illustration of an adenovirus according to the present invention. Modular covalent decoration of the adenovirus capsid via insertion of DogTag into hexon HVR loops. Modular display of DogCatcher-fused ligands on the surface of the adenovirus capsid via covalent coupling with DogTag inserted into hexon HVR surface loops.

FIG. 2(A-B): shows a schematic illustration of construct designs for display on the surface of the adenovirus capsid according to FIG. 1.

FIG. 2A shows the Tag protein (e.g. DogTag) is inserted into the HVR5 loop of a capsid protein (e.g. hexon protein). Here the Tag protein is flanked, both upstream and downstream, by two glycine serine linkers, preferably a GSGGSG linker. The linkers (particularly flexible linkers such as Glycine serine linkers) facilitate and may improve the insertion of the Tag protein into the capsid protein as such a linker increases the flexibility of the region into which the Tag protein is inserted. Increased flexibility may thus improve coupling efficiency. FIG. 2A shows one embodiment of the present invention, in which linkers are inserted on both sides of the Tag protein to maximize flexibility and thus coupling efficiency. However, it will be appreciated that the presence of a linker is optional. One advantage of insertion into a hexon HVR loop, particularly the HVR5 loop, is that these regions are already flexible. As such insertion of the DogTag into the hexon HVR loop (i.e. without any linkers) would be suitably reactive to couple effectively. In addition, other variations of the invention in which the linker is inserted upstream and/or downstream of the Tag protein sequence is envisaged to also be suitable without departing from the invention.

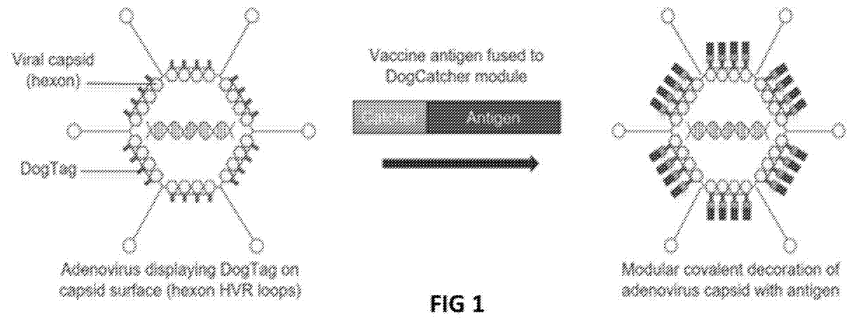

The resulting fusion protein comprises a capsid protein having a Tag protein (e.g. DogTag) in the HVR5 loop. As such, the capsid protein (e.g. hexon protein) component of the fusion protein anchors the first peptide partner (e.g. Tag protein, preferably DogTag) in the viral capsid (as in FIG. 1) so that the first peptide partner (e.g. Tag protein, preferably DogTag) decorates the surface of the viral capsid. This enables the modular attachment of a corresponding second peptide partner (e.g. Catcher protein, preferably DogCatcher) that has been modified (e.g. by fusion) to comprise an antigen of choice. Since the antigen is not directly fused to the viral capsid a greater range of antigens (e.g. size) and a selection of different combinations of antigens being modularly attached to the viral capsid is made possible.

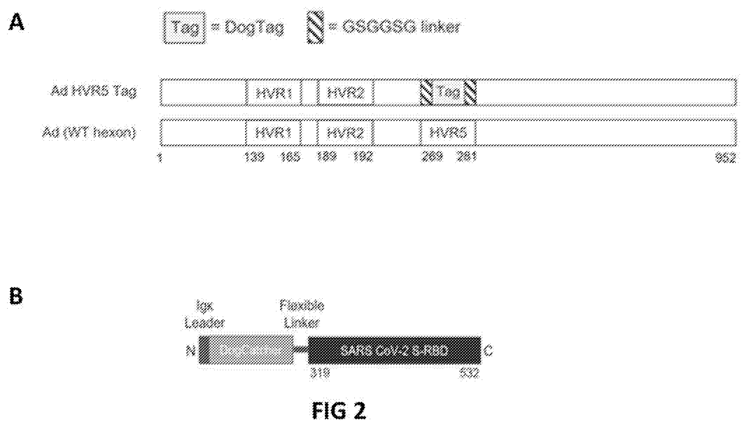

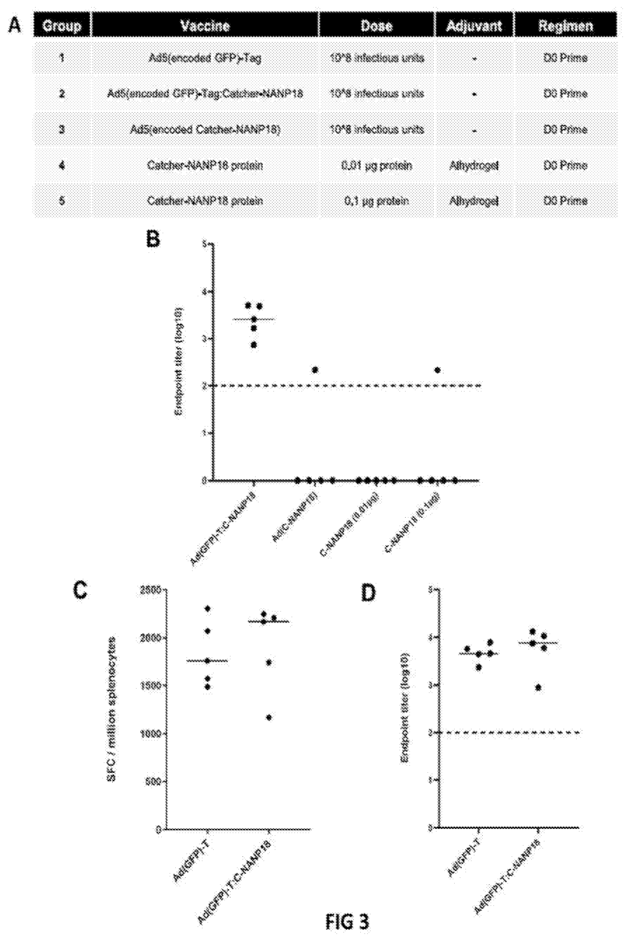

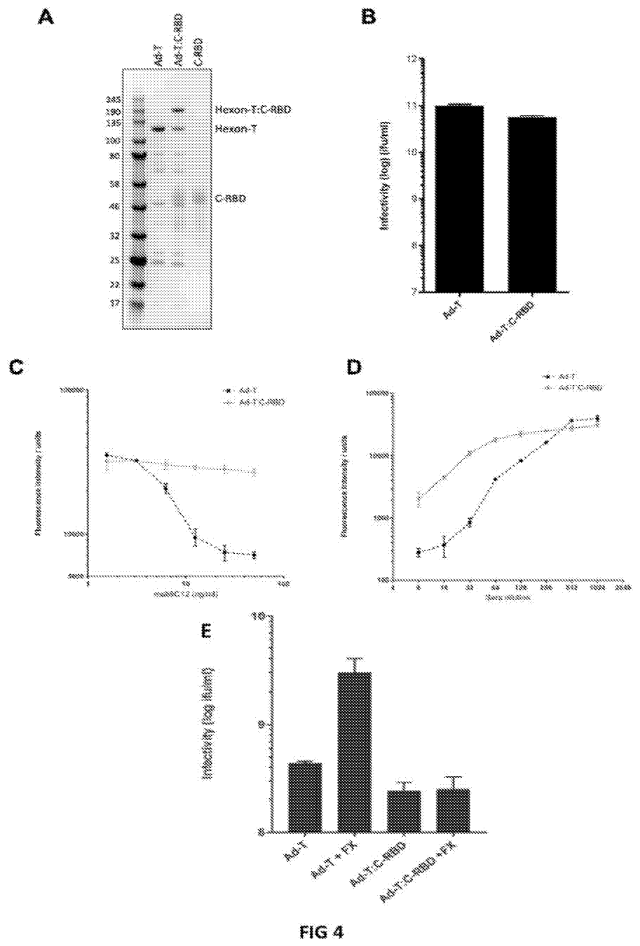

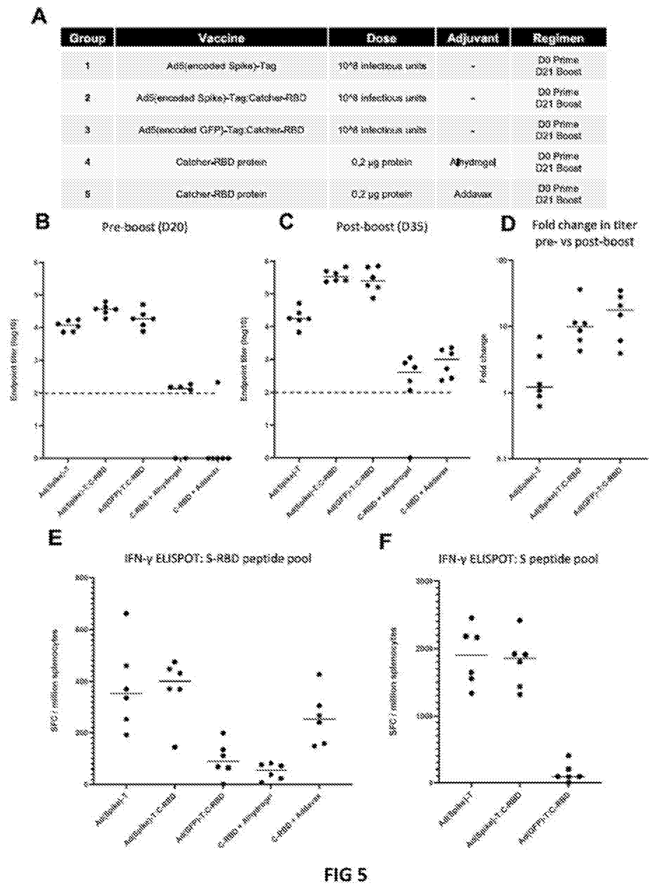

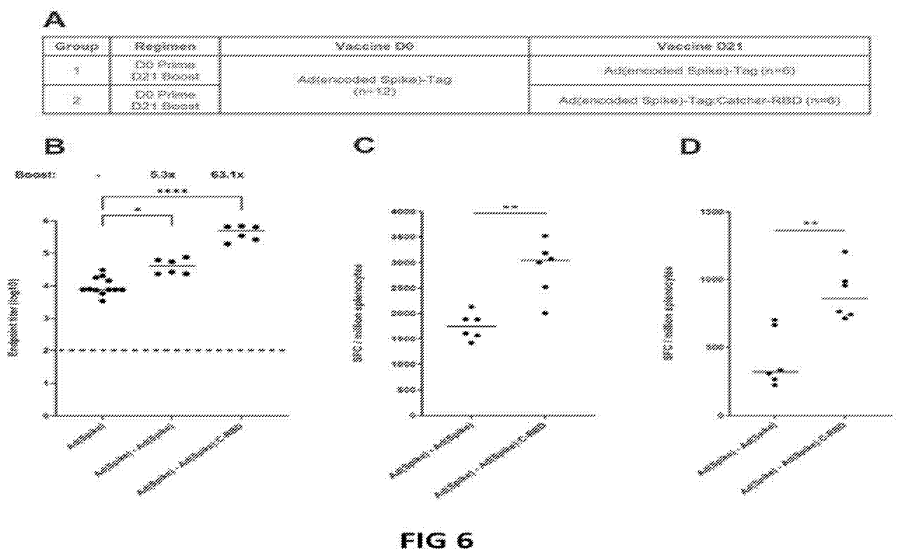

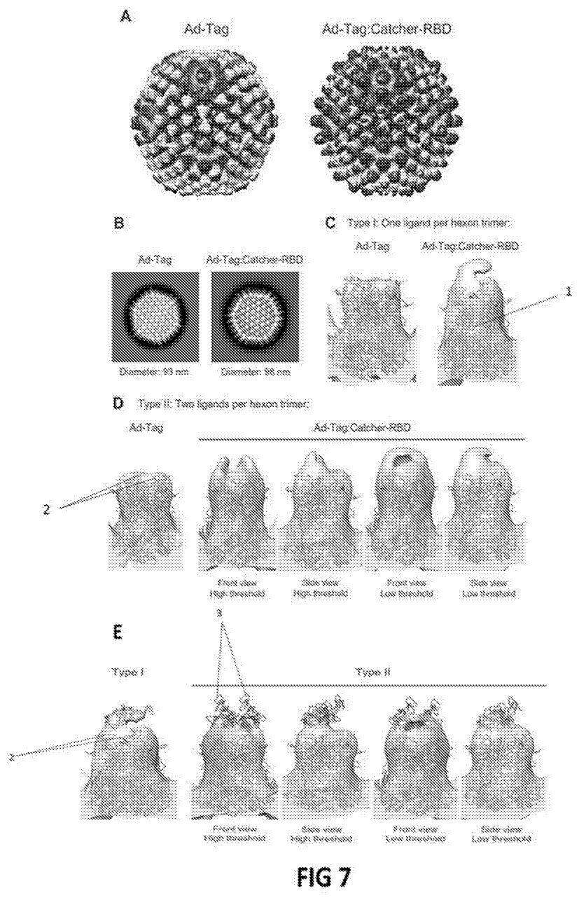

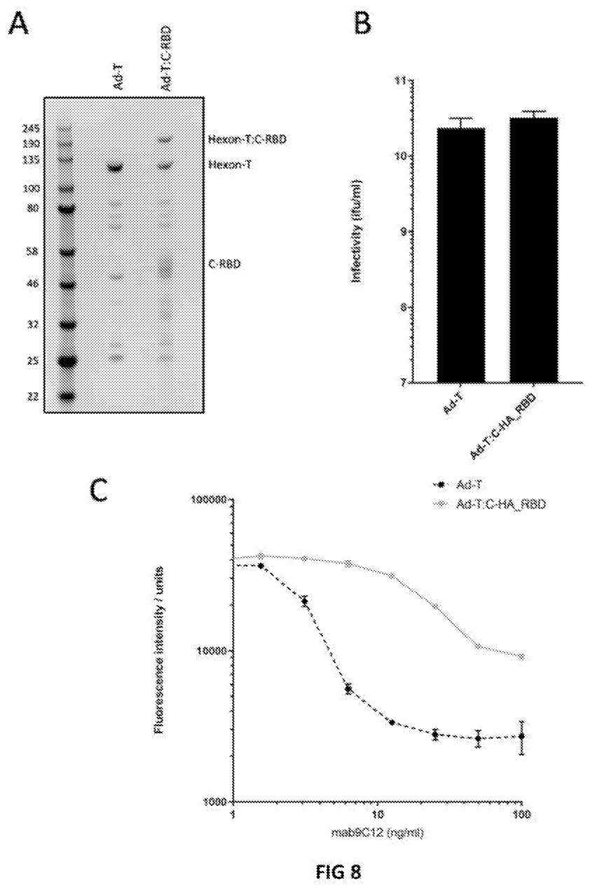

FIG. 2B shows an optional design of DogCatcher-SARS CoV-2-RBD protein for display on the surface of the adenovirus capsid according to FIG. 1. The SARS CoV-2-RBD protein (e.g. antigen) is position towards the C-terminal domain. A flexible linker is shown between the catcher (DogCatcher) and the antigen (e.g., SARS CoV-2 S-RBD protein). Also shown is the inclusion of an IgK leader at the N-terminal domain.

FIG. 3. Antibody responses against Catcher-NANP generated through adenovirus capsid surface display. FIG. 3A: Design and immunization schedule for mouse immunogenicity experiment to assess antibody titers to a capsid displayed antigen (Catcher-NANP18) and T cell and antibody responses to a vector encoded antigen (GFP) with and without a capsid ligand. Balb/c mice (5/group) were immunized intramuscularly with a single dose of either Ad5 encoding GFP with DogTag inserted at HVR5 (Ad(GFP)-T, Group 1), the same vector but with DogCatcher-NANP18 also coupled to the capsid surface (Ad(GFP)-T:C-NANP18, Group 2), Ad5 encoding the DogCatcher-NANP18 construct (Ad(C-NANP18), Group 3), or DogCatcher-NANP18 (C-NANP18) recombinant protein in alhydrogel adjuvant at a dose of 0.01 μg protein (Group 4) or 0.1 μg protein (Group 5). Adenovirus vectors in groups 1-3 were administered at a dose of 10 8 infectious units as calculated by a single cell infectivity assay. During the preparation of the Ad(GFP)-T:C-NANP18 vaccine batch, excess C-NANP18 protein was removed by dialysis after conjugation. The C-NANP18 protein dose in Group 2 was calculated to be less than 0.05 μg per mouse.

FIG. 3B-D: After 14 days post immunization, mice were sacrificed, and humoral and cellular responses to vaccine antigens were measured. FIG. 1B: Serum IgG antibody responses to C-NANP18 in Groups 2-5 were measured by endpoint ELISA. FIG. 1C: CD8+ T cell responses in the spleen to encoded GFP epitope HYLSTQSAL (EGFP200-208) were measured by overnight ex vivo IFNγ-ELISPOT in groups 1 and 2. FIG. 1D: Serum IgG antibody responses to the encoded GFP antigen in Groups 1 and 2 were measured by endpoint ELISA. In FIG. 3B-D, bars show median responses.

FIG. 4. Display of Catcher-SARS CoV-2 S-RBD on the adenovirus capsid provides a shield from anti-vector neutralising antibodies. FIG. 4A-B: Reactivity of DogCatcher-SARS CoV-2 Spike Receptor Binding Domain (S-RBD) with DogTag inserted into hexon HVR5 loop on the Ad5 capsid. Ad5(GFP) vectors (1E+10 viral particles) displaying DogTag at HVR5 were incubated with CHO cell expressed DogCatcher-S-RBD fusion protein at a concentration of 3.5 μM. Reactions were performed overnight (16h) at 4° C.

FIG. 4A: Samples were run on an SDS-PAGE gel and proteins visualized by coomassie staining. Coupling efficiency was assessed calculated by comparing band intensities of unconjugated hexon-Tag in ligand decorated samples to undecorated (control) samples using Image J; an efficiency of 64% hexon coupling was calculated.

FIG. 4B: A vector infectivity assay (by GFP focus enumeration) was performed on the same samples shown in A. Bars display mean and range of duplicate samples.

FIG. 4C: Coupling of DogCatcher-S-RBD (C-RBD) to the Ad5 capsid reduces the potency of a potent vector neutralising monoclonal antibody. Ad5 vectors encoding GFP and either with a C-RBD shield (Ad-T:C-RBD) or without a capsid shield (Ad-T) were added to 293A cells in the presence of a varying concentration of a monoclonal antibody targeting the adenovirus (Ad5) hexon (mAb 9C12). Productive adenovirus infection was detected from the fluorescence of adenovirus-encoded GFP expressed in the cells. Intensity of fluorescence of Ad5-T alone or Ad-T:C-RBD with increasing mAb concentration is shown.

FIG. 4D: Coupling of DogCatcher-S-RBD (C-RBD) to the Ad5 capsid reduces the vector neutralisation potency of polyclonal Ad5 immune sera. Ad5 vectors encoding GFP and either with a C-RBD shield (Ad-T:C-RBD) or without a capsid shield (Ad-T) were added to 293A cells in the presence of varying dilutions of Ad5-neutralising mouse serum. Productive adenovirus infection was detected from the fluorescence of adenovirus-encoded GFP expressed in the cells. Intensity of fluorescence of Ad5-T alone or Ad-T:C-RBD with decreasing concentration of neutralising sera is shown.

FIG. 4E: Capsid decoration with DogCatcher S-RBD impairs human Factor X (FX) mediated Ad transduction of SKOV3 cells. Ad5 vectors encoding GFP (Ad-T), either with or without a DogCatcher-S-RBD (C-RBD) capsid shield were incubated on SKOV3 cells in the presence (+FX) or absence of recombinant human Factor X. Infectivity data (GFP focus assay) show mean+SD of triplicate wells.

FIG. 5. High-titer antibody responses generated against SARS CoV-2 S-RBD when displayed on the adenovirus capsid surface.

FIG. 5A: Design and immunization schedule for mouse immunogenicity experiment to assess antibody titers to SARS CoV-2 S-RBD and T cell responses against SARS CoV-2 S/S-RBD using adenovirus capsid display technology. Balb/c mice (6/group) were immunized intramuscularly with two doses (on Day 0 and Day 21) of either Ad5 encoding SARS CoV-2 Spike with DogTag inserted at HVR5 (Ad(Spike)-T, Group 1), the same Spike encoding vector but with DogCatcher-SARS CoV-2 S-RBD also coupled to the capsid surface (Ad(Spike)-T:C-RBD, Group 2), Ad5 encoding GFP with DogTag inserted at HVR5 and DogCatcher-SARS CoV-2 S-RBD coupled to the capsid surface (Ad(GFP)-T:C-RBD, Group 3) or 0.2 μg DogCatcher-SARS CoV-2 S-RBD (C-RBD) recombinant protein in either Alhydrogel (Group 4) or Addavax (Group 5) adjuvants. Adenovirus vectors in groups 1-3 were administered at a dose of 10 8 infectious units as calculated by a single cell infectivity assay. During the preparation of the Ad(Spike)-T:C-RBD and Ad(GFP)-T:C-RBD vaccine batches, excess C-RBD protein was removed by dialysis after conjugation. The C-RBD protein dose in Groups 2 and 3 was calculated to be less than 0.2 μg per mouse.

FIG. 5B: Endpoint ELISA showing IgG titers against SARS CoV-2 S-RBD in sera from tail vein bleeds performed on Day 20 (pre-boost).

FIG. 5C: Endpoint ELISA showing IgG titers against SARS CoV-2 S-RBD in sera from cardiac bleeds performed two weeks post-boost (Day 35).

FIG. 5D: Fold change in SARS CoV-2 S-RBD IgG ELISA titers between pre- and post-boost samples.

FIG. 5E: IFNγ T cell ELISPOT responses in the spleen against SARS CoV-2 S-RBD peptide pool on Day 35.

FIG. 5F: IFNγ T cell ELISPOT responses in the spleen against SARS CoV-2 Spike peptide pools on Day 35. Responses are the sum of two peptide pools together spanning the full length of the Spike protein.

In FIG. 5A-F, bars show median responses.

FIG. 6: Applying DogCatcher SARS CoV-2 Spike-RBD capsid decoration at boost significantly increases SARS CoV-2 specific antibody and T cell responses in an adenovirus vector prime-boost regimen

FIG. 6A: BALB/c mice (n=12) were immunized intramuscularly with Ad(Spike)-Tag on DO and then on D21 given a second intramuscular immunization of either Ad(Spike)-Tag (Group 1, n=6) or Ad(Spike)-Tag:Catcher-RBD (Group 2, n=6). All vaccines were administered at a dose of 10 8 infectious units. FIG. 6B: Serum IgG antibody responses to RBD measured by endpoint ELISA. Responses measured post prime on D20 (Ad(Spike) are compared to responses on D35 after homologous (Ad(Spike)-Ad(Spike) or heterologous (Ad(Spike)-Ad(Spike):C-RBD) prime boost. Fold change in median titer post boost displayed. Dashed line represents limit of detection. FIG. 6C: IFNγ-ELISPOT response in spleen at D35 against peptide pool spanning full length (1-1273) SARS CoV-2 S. FIG. 6D: IFNγ-ELISPOT response in spleen at D35 against peptide pool spanning C-terminal residues 633-1273 of SARS CoV-2 S only (i.e. not including RBD domain). In B-D, data show median responses.

FIG. 7: Cryo-TEM analysis of adenovirus particles displaying DogCatcher-SARS CoV-2 Spike-RBD

FIG. 7A: 3D density maps (at 10.5 Å) for Ad-DogTag (Ad-Tag, control sample, undecorated) and Ad-DogTag:DogCatcher-RBD (Ad-Tag:Catcher-RBD) particles. Radial colouring scheme is indicated (in greyscale). FIG. 7B: Exemplary 2D class averages. Indicated diameters calculated from vertex to vertex. FIG. 7C: Type I ligand coupling; 3D structure of representative hexon trimer without ligand (Ad-Tag) or with one ligand coupled per trimer (Ad-Tag:Catcher-RBD) shown at the same contour level. FIG. 7D: Type II ligand coupling; 3D structure representative of hexon trimer adjacent to penton base without ligand (Ad-Tag) or with two ligands coupled per trimer (Ad-Tag:Catcher-RBD) shown at the same contour level). Maps for Ad-DogTag: DogCatcher-RBD shown at both front and side angles, and high and low threshold to indicate location and extent of additional electron density. In both 7C and 7D, hexon trimer structure (PDB 6B1T) was fitted, with location of HVR5 loop (residues 270-280, site of DogTag insertion) shown. FIG. 7E CryoEM; fitting S-RBD structure into density maps of Ad-Tag:Catcher-RBD. Structure of the SARS CoV-2 Spike receptor binding domain (S-RBD) (PDB ID J7VB) was fitted into 3D density maps for Type I and Type II ligand coupling to hexon trimers on the surface of Ad-Tag:Catcher-RBD from FIGS. 6C and 6D. Hexon trimer structure (PDB ID 6B1T) shown (as before −1), with location of HVR5 loop (residues 270-280, site of DogTag insertion) shown.

FIG. 8. Display of Catcher-haemagglutinin-RBD on the adenovirus capsid provides a shield from an anti-capsid neutralising antibody.

FIG. 8A-B: Reactivity of DogCatcher fused to Influenza A haemagglutinin receptor binding domain (DogCatcher-HA-RBD) with DogTag inserted into hexon HVR5 loop on the Ad5 capsid. Ad5(GFP) vectors (5E+9 viral particles) displaying DogTag at HVR5 were incubated with CHO cell expressed DogCatcher-HA-RBD fusion protein at a concentration of 1.75 μM. Reactions were performed overnight (16h) at 4° C.

FIG. 8A: Samples were run on an SDS-PAGE gel and proteins visualized by coomassie staining. Coupling efficiency was calculated by comparing band intensities of unconjugated hexon-Tag in ligand decorated samples to undecorated (control) samples using Image J; an efficiency of 44% hexon coupling was calculated.

FIG. 8B: A vector infectivity assay (by GFP focus enumeration) was performed on the same samples shown in A. Bars display mean and standard deviation of triplicate samples.

FIG. 8C: Coupling of DogCatcher-HA-RBD (C-HA_RBD) to the Ad5 capsid reduces the potency of a potent vector neutralising monoclonal antibody. Ad5 vectors encoding GFP and either with a DogCatcher-HA-RBD shield (Ad-T:C-HA_RBD) or without a capsid shield (Ad-T) were added to 293A cells in the presence of a varying concentration of a monoclonal antibody targeting the adenovirus (Ad5) hexon (mAb 9C12). Productive adenovirus infection was detected from the fluorescence of adenovirus-encoded GFP expressed in the cells. Intensity of fluorescence of Ad5-T alone or Ad-T:C-HA_RBD with increasing mAb concentration is shown.

### DETAILED DESCRIPTION

The present disclosure relates to adenoviral vector comprising a transgene encoding an antigen having a T cell epitope. The vector capsid comprising a modified capsid protein having a first peptide partner. A second peptide partner is attached to the first peptide partner to provide a covalently linked peptide binding pair. The second peptide partner also being attached to an antigen, the antigen having a B cell epitope. In a preferred embodiment the transgene encoding one antigen is in the lumen of the viral capsid and the second peptide attached to the second antigen is displayed on the surface of the viral capsid. This display may be achievable by means of a fusion protein formed from a capsid protein and a first peptide partner, said first peptide partner being decorated on the surface of the viral capsid. Further aspects of the invention relate to vaccines comprising said vector, its use in therapy and methods of manufacture and treatment thereof.

The invention will be more clearly understood from the following description of some embodiments thereof, given by way of example only, with reference to the Figures and tables where appropriate.

All publications, patent applications, patents and other references mentioned are incorporated by reference in their entirety.

This disclosure is not limited to any particular embodiments described, as such the terminology used herein is for the purpose of describing particular embodiments only, and is not intended to be limiting in any way.

### Definitions

In order for the present disclosure to be more readily understood, certain terms employed herein are collected and first defined below. Additional definitions for subsequent terms are set forth throughout the specification. Unless otherwise defined, all other technical and scientific terms used herein have the commonly understood meaning of someone of ordinary skill in the art to which this disclosure is related.

As used in the specification and claims, the singular form “a”, “an” and “the” include plural references unless the context clearly dictates otherwise. For example, the term “a cell” includes a plurality of cells, including mixtures thereof.

All numerical designations, e.g., pH, temperature, time, concentration, and molecular weight, including ranges, are approximations which are varied (+) or (−) by increments of 0.1. It is to be understood, although not always explicitly stated, that all numerical designations are preceded by the term “about.” It also is to be understood, although not always explicitly stated, that the reagents described herein are merely examples and that equivalents of such are known in the art.

The term “and/or” where used herein is to be taken as specific disclosure of each of the two specified features or components with or without the other. Thus, the term “and/or” as used in a phrase such as “A and/or B” herein is intended to include A and B; A or B; A (alone); and B (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “e.g.,” and “i.e.” as used herein, are used merely by way of example, without limitation intended, and should not be construed as referring only those items explicitly enumerated in the specification.

The terms “or more”, “at least”, “more than”, and the like, e.g., “at least one” are understood to include but not be limited to at least 1, 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more than the stated value. Also included is any greater number or fraction in between.

Conversely, the term “no more than” includes each value less than the stated value. For example, “no more than 100 nucleotides” includes 100, 99, 98, 97, 96, 95, 94, 93, 92, 91, 90, 89, 88, 87, 86, 85, 84, 83, 82, 81, 80, 79, 78, 77, 76, 75, 74, 73, 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, and 0 nucleotides. Also included is any lesser number or fraction in between.

The terms “plurality”, “at least two”, “two or more”, “at least second”, and the like, are understood to include but not limited to at least 2, 3, 4, 5, 6, 7, 8, 9, 10, 11, 12, 13, 14, 15, 16, 17, 18, 19 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150, 200, 300, 400, 500, 600, 700, 800, 900, 1000, 2000, 3000, 4000, 5000 or more. Also included is any greater number or fraction in between.

Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. It is understood that wherever aspects are described herein with the language “comprising,” otherwise analogous aspects described in terms of “consisting of” and/or “consisting essentially of” are also provided. The term “consisting of” excludes any element, step, or ingredient not specified in the claim. In re Gray, 53 F.2d 520, 11 USPQ 255 (CCPA 1931); Ex parte Davis, 80 USPQ 448, 450 (Bd. App. 1948) (“consisting of” defined as “closing the claim to the inclusion of materials other than those recited except for impurities ordinarily associated therewith”). The term “consisting essentially of” limits the scope of a claim to the specified materials or steps “and those that do not materially affect the basic and novel characteristic(s)” of the claimed invention.

Unless specifically stated or evident from context, as used herein, the term “about” refers to a value or composition that is within an acceptable error range for the particular value or composition as determined by one of ordinary skill in the art, which will depend in part on how the value or composition is measured or determined, i.e., the limitations of the measurement system. For example, “about” or “approximately” may mean within one or more than one standard deviation per the practice in the art. “About” or “approximately” may mean a range of up to 10% (i.e., +10%). Thus, “about” may be understood to be within 10%, 9%, 8%, 7%, 6%, 5%, 4%, 3%, 2%, 1%, 0.5%, 0.1%, 0.05%, 0.01%, or 0.001% greater or less than the stated value. For example, about 5 mg may include any amount between 4.5 mg and 5.5 mg. Furthermore, particularly with respect to biological systems or processes, the terms may mean up to an order of magnitude or up to 5-fold of a value. When particular values or compositions are provided in the instant disclosure, unless otherwise stated, the meaning of “about” or “approximately” should be assumed to be within an acceptable error range for that particular value or composition.

As described herein, any concentration range, percentage range, ratio range or integer range is to be understood to be inclusive of the value of any integer within the recited range and, when appropriate, fractions thereof (such as one-tenth and one-hundredth of an integer), unless otherwise indicated.

Units, prefixes, and symbols used herein are provided using their Système International de Unites (SI) accepted form. Numeric ranges are inclusive of the numbers defining the range.

### Adenovirus

Adenoviruses (Ad) are a non-enveloped double stranded DNA virus with a genome of approximately 36 kilobases (kb). There are over 60 human adenovirus serotypes grouped into species A-G. Each group comprises of a number of adenoviral serotypes, for example, the subgroup species C includes Ad5 and Ad2. Ad5 is the most extensively studied serotype, and the most widely used platform for the development of oncolytic viruses. In the development of oncolytic viruses, it is desirable to be able to target particular tissues, and therefore the tropism may be altered. A major issue with using some adenovirus serotypes, including Ad5, in clinical settings is the pre-existing immunity in humans. Some scientist have tried to overcome the pre-existing immunity by using adenovirus first isolated from the great apes, such as Gorilla and Chimpanzees. Examples of such vectors that have been used in clinical studies include ChAd3, ChAd155, and ChAdOx1. Such adenoviruses are hoped to have a lesser sero-prevalence in humans. Nonetheless humans often have pre-existing to such viruses and of course the issue of pre-existing immunity should it be desired to administer the vector on more than one occasion.

Adenoviruses are typically 70-90 nm in size with an icosahedral capsid shape. The outer capsid structure, also known as ‘capsid protein’ comprises three major types of protein (hexon, fiber and penton base). There are additional minor proteins in the outer capsid including VI, VIII, IX, IIIa and IVa2. Hexon is the major component of the adenoviral capsid accounting for more than 83% of the capsid protein https://www.ncbi.nlm.nih.gov/pmc/articles/PMC187380/. Hexon modification has been shown to allow for circumvention of pre-existing neutralising antibodies in some circumstances, including the swapping of hyper variable regions (HVR) from different serotypes https://www.nature.com/articles/nature04721?proof=t.

### Adenovirus for Modification

Adenovirus can be replication-defective: certain genes are deleted from the genome in order to ensure that when the adenovirus is used as a therapeutic, it is no longer capable of replication. For vaccination purposes these vectors typically have deletions in E1 (renders vector replication defective) and/or E3 genes (increases capacity for insert size). Other vectors may result from the deletion of a set of genes from the genome, and is within the skills of those working with adenoviruses. This is an advantage for use in vaccines, where the aim of the adenoviral vector is to present the antigen to the immune system in a format that makes it highly immunogenic, while limiting cytotoxicity.

The adenovirus may be from any serotype or strain of adenovirus. Therefore, suitable adenoviruses for modification may come from those that infect mammals other than humans, in order to minimise prior exposure effects. The capsid structure is strongly conserved, and therefore the adenoviral serotypes and species may be interchangeable.

The adenovirus may be any modified adenovirus. Thus, the modified adenovirus encodes antigens. These encoded antigens are expressed after transduction. This provides the possibility of a multi-faceted prophylactic or therapeutic, such that an antigen containing at least one B cell epitope can be displayed on the surface of the virus and another antigen expressed upon vector transduction using host cell machinery. Thus, the adenovirus is genetically modified, such that it includes a transgene. This transgene is designed for delivery to the host cell and encodes an antigen containing at least one T cell epitope.

### Adenovirus-Mediated Infectivity

Adenovirus infectivity in cells that express the Coxsackievirus and adenovirus receptor (CAR) is mediated via the fiber protein. An example of a cell line that expresses the CAR receptor is HEK293 cells. Fiber binds to the CAR receptor on the surface of cells and this mediates the initial attachment of the virus. However, it was recently demonstrated that instead of a fiber-mediated entry of the adenovirus, Factor X (FX)—a coagulation factor present in human serum can bind to the hexon proteins of some adenovirus serotypes to facilitate the entry of the virus in some cell types. An example of a cell line that mediates infection via the hexon protein is SKOV3. It is believed that FX mediated infection via the adenovirus hexon can enhance liver tropism of adenovirus vectors in vivo. Modifications of the hexon protein such as insertion of DogTag and coupling to an antigen reduces hexon-mediated infectivity of the cells. This is a desirable effect as the natural tropism of adenovirus when injected intravenously can cause liver toxicity in patients at very high doses. Reduction of hexon-mediated infectivity to reduce liver toxicity would be advantageous to the present invention.

### Hexon Capsid Protein

The hexon capsid protein is approximately 100 kDa in size, with 720 monomers per virion. Hexon monomers organise into trimers so that 12 lie on each of the 20 facets, resulting in 240 trimers per virion. Hexon sequences contain hypervariable regions (HVR) corresponding to loops on the external surface on the virus and therefore cover almost the entire surface of the virus. Each monomer has seven HVRs identified as HVR1-HVR7 which are serotype specific. As the loops are on the external surface of the virus, hexon loops are the main antigen recognition site, a target for host immune responses. Hexon protein varies in length, for example, Ad2 is the longest known hexon protein with a length of 968 amino acids (UniProt ID: P03277). Ad5, the most commonly used adenovirus for gene therapy has a length of 952 amino acids (UniProt ID: P04133). Modifying hexon HVRs which contain the serotype-specific epitope seems to be a promising approach to overcome the host neutralisation response. Any one of the HVRs could be modified. Exemplified herein, modifications were successfully made to HVR5, surprisingly using DogTag. When the hexon protein was modified according to the invention and an antigen was attached via the peptide partner pair, neutralisation by anti-adenovirus neutralising antibodies was reduced.

pIX Capsid Protein pIX protein is a minor capsid protein which is approximately 14.3 kDa in size. There are approximately 240 pIX monomers per virion. The pIX protein functions to stabilise the hexons on the viral surface. The C-terminus of the pIX protein is exposed on the surface of the virus and is therefore a desirable site for fusion of small and large peptides. Ad5 pIX has two domains connected by a flexible linker. The Ad5 pIX protein has a length of 196 amino acids (UniProt ID: Q2KS03).

### Modifications to the Capsid Proteins

Modification to the capsid proteins can be genetic or non-genetic, including chemical and/or genetic/protein engineering. Chemical modification would typically be performed once the viral capsid had already been formed. Engineering approaches typically involve a premeditated change of the amino acid sequence and/or the corresponding nucleic acid sequence (i.e. genetic modification) so as to influence the properties of the protein (e.g., efficiency, binding capabilities, stereoselectivity, shielding etc.). As such proteins may be engineered to contain insertions, deletions, mutations and/or substitutions in the amino acid residues. The capsid proteins can be genetically modified through the incorporation of antigens into the capsid. Alternatively, the viral particle surface may be directly modified. Modification of all three major capsid proteins has been demonstrated previously. However, the results from these modifications has been mixed, and there is a major obstacle in the size of the insert that the most promising approaches offer, particularly regarding modification of hexon. For example, HVR loops can only take small insertions (about ≤100 amino acid residues) before the structure of the hexon becomes distorted and the virus can no longer be generated, e.g. where the antigen/ligand (e.g. RBD) is attached (e.g. fused) directly to the hexon protein. So, ligands such as RBD cannot be directly inserted into the hexon protein as they are too big. Chemical modification on the other hand is less precise than genetic modification, is more complex to perform, and may reduce infectivity of the modified virus.

“At least one modification” as used herein refers to the inclusion of a first peptide partner insertion into the viral capsid protein using any appropriate means. For example, the insertion of the first peptide partner into the adenoviral hexon loops or the fusion of a first peptide partner to the adenoviral pIX minor capsid protein. This modification may be made genetically through gene fusion, for example, or chemically.

The term “fusion protein” or “chimeric protein” or “hybrid protein” can refer to a single polypeptide formed from the fusion of two separate proteins or domains, with or without an additional linker sequence and that are encoded for by two separate genes and/or nucleic acid sequences (e.g. gene fusion). There are three main types of fusion protein, end-to-end fusion, insertional fusion and branched fusion (see ‘Methods in Enzymology’—introduction (2021)). “End-to-end fusion protein(s)” are formed by the N-terminal end of the downstream domain linked to the C-terminal end of the upstream domain. “Insertional fusion protein(s)” are formed when a ‘guest domain’ is inserted into the middle of a ‘host domain’. Such fusion proteins are more sensitive to changes in the other domains that form part of the fusion protein and more engineering effort is required. “Branched fusion protein(s)” provide a non-linear approach to fusion and more than two domains are linked together about a central point. Such fusion proteins cannot be genetically encoded for at a DNA level and so are formed at a protein level. End-to-end fusion is the most widely used fusion protein construction.

Typically, formation of a fusion protein may involve the joining together of at least two different nucleic acid sequences or genes (typically referred to as “genetic fusion”) so that when they are transcribed (or expressed) and translated as a single unit to produce a single polypeptide comprising said separate proteins or domains. Typically, from 1-3 amino acids may be added, deleted or substituted upstream and/or downstream of the fusion site (see below definition) between said separate proteins in the single polypeptide. Although it should be understood that the number of amino acids that may be added, deleted or substituted may depend on the types of proteins making up the single polypeptide and/or the expression and/or translation processes involved. Genetic fusion may also typically involve removal of the stop codon from a cDNA sequence coding for the first protein, then appending the cDNA sequence of the second protein in frame. Said genetic fusion construct (i.e. lacking a stop codon) will then be expressed as a single protein. The protein can be engineered to include the full sequence of both components, or only a portion of each.

One example of a fusion protein, within the scope of the present invention, is hexon protein: DogTag.

The term “fusion site” refers the interface between two proteins (or domains), where the two proteins link together to form a single polypeptide (domain 1 —fusion site—domain 2).

The modified capsid proteins of the present invention may include a linker, spacer and/or scaffold sequence, the presence of which enables flexibility, stability and optimal conformational display of the Tag protein (e.g., DogTag) in/on the capsid protein.

The term “linker” or “linker sequence” is a sequence, typically an amino acid sequence used to fuse, link or join two proteins (see ‘Methods in Enzymology’, 2021), herein incorporated by reference. The characteristics of linkers and their suitability for particular purposes are known in the art. See, e.g., Chen et al. Adv Drug Deliv Rev. October 15; 65(10): 1357-1369 (2013) (disclosing various types of linkers, their properties, and associated linker designing tools and databases), which is incorporated herein by reference. In some embodiments, the linker is flexible, rigid, or in vivo cleavable. In some embodiments, the linker is flexible. Flexible linkers typically comprise small non-polar amino acids (e.g. Gly) or polar amino acids (e.g., Ser or Thr). Examples of flexible linkers that can be used in the present disclosure include are sequences consisting primarily of stretches of glycine (G)n wherein for example n=1-6, stretches of serine (S)n wherein for example n=1-6, or stretches of glycine and serine residues (“GxS linker”) linkers wherein for example x=1-4. In the present disclosure flexible linkers may comprise repeats of 4 Gly and Ser residues. The flexible linker may comprise 1-5 repeats of five Gly and Ser residues. Non-limiting examples of flexible linker include (Gly-Gly-Gly-Gly-Ser)n (SEQ ID NO: 39), (Ser-Ser-Ser-Ser-Gly)n, Gly-Ser-Ser-Gly-Gly)n, and Gly-Gly-Ser-Gly-Gly)n, where n may be any integer between 1 and 5. In some embodiments, the linker is between 5 and 25 amino acid residues long. In some embodiments, the flexible linker comprises 5, 10, 15, 20, or 25 residues. In particular the linker may comprise GGGGS (G4S). Other linkers may include rigid linkers such as (EAAAK)n wherein for example n=1-4 (see Arai et al.), A(EAAAK)4ALEA(EAAAK)4A, hybrid linkers such as (GSG)6A(EAAAK)6A(GSG)6A(EAAAK)6A(GSG)6, bending linkers and pro-rich sequences (XP)n wherein for example X designates and amino acid (e.g., Ala, Lys, Glu . . . ), or cleavable linkers such as di-sulphide and/or protease sensitive sequences. Including a linker may help to control the spatial and functional relationships between two or more domains by providing distance between the domains, while keeping them connected (See ‘Methods in Enzymology’—introduction, 2021) herein incorporated by reference. Other suitable linkers may be selected from the group consisting of AS, AST, TVAAPS, TVA, ASTSGPS, KESGSVSSEQLAQFRSLD, EGKSSGSGSESKST, (Gly)6, (Gly)8, and GSAGSAAGSGEF. In general, a flexible linker provides good flexibility and solubility and may serve as a passive linker to keep a distance between functional domains. The length of the flexible linkers can be adjusted to allow for proper folding or to achieve optimal biological activity of the fusion proteins. In some embodiments, the linker comprises the sequence (Gly-Gly-Gly-Gly-Ser). In the present disclosure the fusion protein may comprise more than one linker. The fusion protein may have a first and a second linker, wherein the first and second linkers may comprise the same sequences or alternatively may comprise different sequences.

### Peptide Partner Pairs

Proteins that are capable of spontaneous isopeptide bond formation (so-called “isopeptide proteins”) have been advantageously used to develop peptide partner pairs (i.e. two-part linkers) which covalently bind to each other and provide irreversible interactions (see e.g. WO2011/098772 and WO 2016/193746 both herein incorporated by reference, together with WO2018/189517 and WO2018/197854 both incorporated herein by reference). In this respect, proteins which are capable of spontaneous isopeptide bond formation may be expressed as separate fragments, to give a first peptide partner and a second peptide partner which is the peptide binding partner for the first peptide partner, where the two fragments are capable of covalently reconstituting by isopeptide bond formation. This covalent reconstitution links molecules or components fused to the second peptide partner and the requisite first peptide partner. The isopeptide bond formed by the peptide partner pair is stable under conditions where non-covalent interactions would rapidly dissociate, e.g. over long periods of time (e.g. weeks), at high temperature (to at least 95° C.), at high force, or with harsh chemical treatment (e.g. pH 2-11, organic solvent, detergents or denaturants).

Isopeptide bonds are amide bonds formed between carboxyl/carboxamide and amino groups, where at least one of the carboxyl or amino groups is outside of the protein main-chain (the backbone of the protein). Such bonds are chemically irreversible under typical biological conditions and they are resistant to most proteases. As isopeptide bonds are covalent in nature, they result in some of the strongest measured protein-protein interactions.

In brief, a two-part linker, i.e. a peptide partner pair (a so-called peptide tag/binding partner or catcher pair) may be derived from a protein capable of spontaneously forming an isopeptide bond (an isopeptide protein), wherein the domains of the protein are expressed separately to produce a peptide “tag” that comprises one of the residues involved in the isopeptide bond (e.g. an aspartate or asparagine, or a lysine) and a peptide or peptide binding partner (or “catcher”) that comprises the other residue involved in the isopeptide bond (e.g. a lysine, or an aspartate or asparagine) and at least one other residue required to form the isopeptide bond (e.g. a glutamate). Mixing the peptide tag and binding/catcher partner results in the spontaneous formation of an isopeptide bond between the tag and binding partner. Thus, by separately incorporating the peptide tag and binding partner into different molecules or components, e.g. proteins, it is possible to covalently link said molecules or components together via an isopeptide bond formed between the peptide tag and binding partner, i.e. to form a linker between the molecules or components incorporating the peptide tag and binding partner.

The spontaneous formation of the isopeptide bond may be in isolation, and not require the addition of any other entity. For some peptide tag and binding/catcher partner pairs, the presence of a third or helper entity, such as a ligase, may be required in order to generate the isopeptide bond.

A peptide tag/binding partner pair (two-part linker), termed SpyTag/SpyCatcher, has been derived from the CnaB2 domain of the Streptococcus pyogenes FbaB protein (Zakeri et al., 2012, Proc Natl Acad Sci USA 109, E690-697) and used in diverse applications including vaccine development (Brune et al., 2016, Scientific reports 6, 19234; Thrane et al., 2016, Journal of Nanobiotechnology 14, 30).

Variants, derivatives and modifications of the binding pairs may be made by any suitable means. Variants, derivatives and functionally operative modifications may involve amino acid additions, substitutions, alterations or deletions that retain the same function in relation to the ability to form an isopeptide bond with the relevant binding partner.

For some of the binding pairs, mediation by a third entity such as an enzyme is required. For example, SnoopLigase may be used to mediate the bond formation between SnoopTagJr/SnoopTag and DogTag (Buldun et al, Journal of the American Chemical Society 2018 140 (8), 3008-3018 https://pubs.acs.org/doi/10.1021/jacs.7b13237, incorporated by reference]. Thus, the pairing may require the assistance of an enzyme such as a ligase.

It will be understood that as used herein, either the first peptide partner or the second peptide partner may be the peptide “tag” and the other is the “binding partner/catcher”.

Suitably, the first and second peptide partners form the peptide partner pair termed SpyTag/SpyCatcher. Suitably, the SpyCatcher component is DeltaN1 (AN1) SpyCatcher (as described in Li, L., Fierer, J. O., Rapoport, T. A. & Howarth, M. Structural analysis and optimization of the covalent association between SpyCatcher and a peptide Tag. J. Mol. Biol. 426, 309-317 (2014)) which has a 23 amino acid truncation at the N-terminus compared to “SpyCatcher”.

In other embodiments, the first and second peptide partners form a peptide partner pair which is a mutated version of SpyTag/SpyCatcher displaying an increased rate of reaction for isopeptide bond formation such as, for example, those described in co-pending application, WO2018/197854 and in Keeble et al, PNAS Dec. 26, 2019 116 (52) 26523-26533 https://www.pnas.org/content/116/52/26523.long. In some embodiments, these mutated forms may be useful for the attachment of large proteins (e.g. >50 kDa or >100 kDa, such as the >160 kDa HCMV pentameric protein exemplified herein) and/or where slow reactions or steric hindrance may be an issue.

In embodiments, the first and second peptide partners form a peptide partner pair which is a modified version of RrgACatcher/RrgATag named DogCatcher/DogTag. These latter entities are described in. Keeble et al, Cell Chemical Biology, July 2021 (https://doi.org/10.1016/j.chembiol.2021.07.005). This paper is incorporated by reference.

In other embodiments, the isopeptide proteins forming the peptide partner pair may include SnoopTag/SnoopCatcher, described, for example in WO 2016/193746.

In some embodiments, one or both of the isopeptide proteins forming the peptide partner pair may have N- or C-terminal truncations, whilst still retaining the reactivity of the isopeptide bond.

SdyTag and SdyCatcher were constructed based on the native Cna protein B-type (CnaB) domain from a related fibronectin-binding protein in Streptococcus dysgalactiae. In some embodiments, the isopeptide proteins forming the peptide partner pair are SdyTag and SdyCatcher, or are based on modifications to these peptide partner pairs. Known modifications to the SdyCatcher include the modifications to form QueenCatcher, Mooncake and Katl. Such modified versions of SdyCatcher may be paired with pre-existing or modified tags, including but not limited to SdyTag, SnoopTag, SpyTag, RumTag, RumTrunkTag, Clib9, PhoTag, EntTag or BacTag. Such modifications are described in WO2021/224451 herein incorporated by reference.

Exemplary first and second peptide partner pairs (peptide tag/binding partner pairs; reactive pairs) are described in the following table, this list is not exhaustive and further peptide partner pairs may be suitable for use in the present invention:

Reactive pairs

| | | |
|---|---|---|
| (a) | SpyTag | SpyCatcher |
| | SpyTag002 | |
| | SpyTag002 RG T3H | |
| | SpyTag003 | |
| (b) | SpyTag | SpyCatcher002 |
| | SpyTag002 | |
| | SpyTag002 RG T3H | |
| | SpyTag003 | |
| (c) | SpyTag | SpyCatcher002 D5A A92P Q100D |
| | SpyTag002 | |
| | SpyTag002 RG T3H | |
| | SpyTag003 | |
| (d) | SpyTag | SpyCatcher003 |
| | SpyTag002 | |
| | SpyTag002 RG T3H | |
| | SpyTag003 | |
| (e) | SnoopTag | SnoopCatcher |
| | SnoopTagJr | |
| (f) | RrgATag | RrgACatcher - Modified to form |
| | RrgATag2 | DogCatcher, and DogCatcher (all |
| | DogTag | versions) |
| (g) | Isopeptag | Pilin-C |
| (h) | Isopeptag-N | Pilin-N |
| (i) | PsCsTag | PsCsCatcher |
| (j) | SnoopTagJr | DogTag [mediated by SnoopLigase] |
| | SnoopTag | |
| (k) | SdyTag | SdyCatcher |

These entities are described, for example, in WO2011/098772, WO2016/193746, WO2018/197854, WO2018/189517 or Li L., et al., J. Mol. Biol. 426, 309-317 (2014). All are incorporated by reference.

Exemplary sequences for various peptide partners are listed below:

| Peptide partner | Exemplary Sequence |
|---|---|
| SpyCatcher (SEQ ID | VDTLSGLSSEQGQSGDMTIEEDSATHIKFSKRDEDGKELAGATMELRDSSGKTISTWISDG |
| NO. 1) | QVKDFYLYPGKYTFVETAAPDGYEVATAITFTVNEQGQVTVNGKATKGDAHI |
| SpyCatcher | DSATHIKFSKRDEDGKELAGATMELRDSSGKTISTWISDGQVKDFYLYPGKYTFVETAAPD |
| AN1 (SEQ ID NO. 2) | GYEVATAITFTVNEQGQVTVNGKATKGDAHI |
| SpyCatcher002 (SEQ | VTTLSGLSGEQGPSGDMTTEEDSATHIKFSKRDEDGRELAGATMELRDSSGKTISTWISDG |
| ID NO. 3) | HVKDFYLYPGKYTFVETAAPDGYEVATAITFTVNEQGQVTVNGEATKGDAHT |
| Spycatcher003 (SEQ | VTTLSGLSGEQGPSGDMTTEEDSATHIKFSKRDEDGRELAGATMELRDSSGKTISTWISDG |
| ID NO. 4) | HVKDFYLYPGKYTFVETAAPDGYEVATPIEFTVNEDGQVTVDGEATEGDAHT |
| SpyCatcher | DSATHIKFSKRDEDGKELAGATMELRDSSGKTISTWISDGQVKDFYLYPGKYTFVETAAPD |
| AN1 AC2 (SEQ ID NO. 5) | GYEVATAITFTVNEQGQVTVNG |
| SnoopCatcher (SEQ | KPLRGAVFSLQKQHPDYPDIYGAIDQNGTYQNVRTGEDGKLTFKNLSDGKYRLFENSEPA |
| ID NO. 6) | GYKPVQNKPIVAFQIVNGEVRDVTSIVPQDIPATYEFTNGKHYITNEPIPPK |
| Spyligase (SEQ ID NO. | DYDGQSGDGKELAGATMELRDSSGKTISTWISDGQVKDFYLYPGKYTFVETAAPDGYEVA |
| 7) | TAITFTVNEQGQVTVNGKATKGGSGGSGGSGEDSATHI* |
| SdyCatcher (SEQ ID | LSGETGQSGNTTIEEDSTTHVKFSKRDANGKELAGAMIELRNLSGQTIQSWISDGTVKVFY |
| NO. 8) | LMPGTYQFVETAAPEGYELAAPITFTIDEKGQIWVDS |
| RrgACatcher (SEQ ID | KLGDIEFIKVNKNDKKPLRGAVFSLQKQHPDYPDIYGAIDQNGTYQNVRTGEDGKLTFKNL |
| NO. 9) | SDGKYRLFENSEPAGYKPVQNKPIVAFQIVNGEVRDVTSIVPQ |
| DogCatcher V1 (SEQ | KLGEIEFIKVDKTDKKPLRGAVFSLQKQHPDYPDIYGAIDQNGTYQDVRTGEDGKLTFTNL |
| ID NO. 10) | SDGKYRLIENSEPPGYKPVQNKPIVSFRIVDGEVRDVTSIVPQ |
| DogCatcher V2 (SEQ | KLGEIEFIKVDKTDKKPLRGAVFSLQKQHPDYPDIYGAIDQNGTYQDVRTGEDGKLTFTNL |
| ID NO. 11) | SDGKYRLFENSEPPGYKPVQNKPIVAFQIVDGEVRDVTSIVPQ |
| PsCsCatcher (SEQ ID | EQDVVFSKVNVAGEEIAGAKIQLKDAQGQVVHSWTSKAGQSETVKLKAGTYTFHEASAP |
| NO. 12) | TGYLAVTDITFEVDVQGKVTVKDANGNGVKAD |
| PilinC (SEQ ID NO. 13) | ATTVHGETVVNGAKLTVTKNLDLVNSNALIPNTDFTFKIEPDTTVNEDGNKFKGVALNTP |
| | MTKVTYTNSDKGGSNTKTAEFDFSEVTFEKPGVYYYKVTEEKIDKVPGVSYDTTSYTVQV |
| | HVLWNEEQQKPVATYIVGYKEGSKVPIQFKNSLDSTTLTVKKKVSGTGGDRSKDFNFGLT |
| | LKANQYYKASEKVMIEKTTKGGQAPVQTEASIDQLYHFTLKDGESIKVTNLPVGVDYVVT |
| | EDDYKSEKYTTNVEVSPQDGAVKNIAGNSTEQETSTDKDMTI |
| QueenCatcher (SEQ | IDTMSGLSGETGQSGNTTIEEDSTTHVKFSKRDSNGKELAGAMIELRNLSGQTIQSWVSD |
| ID NO. 14) | GTVKDFYLMPGTYQFVETAAPEGYELAAPITFTVNEQGQVTVNGKATKGDAHI |
| Kat I (SEQ ID NO. 15) | DTMSGLSGETGQSGNTTIEEDSTTHVKFSKRDSNGKELAGAMIELRNLSGQTIQSWVSD |
| | GTVKDFYLMPGTYQFVETAAPEGYELAAPITFTVQDNGEVQIQGKATRGDVPI |
| Mooncake (SEQ ID | IDTMSGLSGETGQSGNTTIEEDSTTHVKFSKRDSNGKELAGAMIELRNLSGQTIQSWVSD |
| NO. 16) | GTVKDFYLMPGTYQFVETAAPEGYELAAPITFTVQDNGEVIIQGRLTRGDVHI |
| SpyTag (SEQ ID NO. 17) | AHIVMVDAYKPTK |
| SpyTag002 (SEQ ID | VPTIVMVDAYKRYK |
| NO. 18) | |
| SpyTag003 (SEQ ID | RGVPHIVMVDAYKRYK |
| NO. 19) | |
| SnoopTag (SEQ ID | KLGDIEFIKVNK |
| NO. 20) | |
| SnoopTag Jr (SEQ ID | KLGSIEFIKVNK |
| NO. 21) | |
| DogTag (SEQ ID NO. 22) | DIPATYEFTDGKHYITNEPIPPK |
| SdyTag (SEQ ID NO. 23) | DPIVMIDNDKPIT |
| PsCsTag (SEQ ID NO. 24) | GNKLTVTDQAAPS |
| RrgATag (SEQ ID NO. 25) | DIPATYEFTNDKHYITNEP |
| IsopepTag (SEQ ID | TDKDMTITFTNKKDAE |
| NO. 26) | |
| RumTag (SEQ ID NO. 27) | AGCGAAAACGGCAACCCGCTGATTGTGATGGTGGATGATACCACCAAAGTGAAAATT |
| | AGC |
| Clib9 (SEQ ID NO. 28) | RGVPTIVMVDCYKRYK |
| RumTrunkTag (SEQ | GNPLIVMVDDTTKVK |
| ID NO. 29) | |
| RumTrunk D9N Tag | GNPLIVMVNDTTKVK |
| (SEQ ID NO. 30) | |
| PhoTag (SEQ ID NO. 31) | LVTGTAHIVMVDNYKPIVETGD |
| EntTag (SEQ ID NO. 32) | NTIVMVDKLKEVPPT |
| Rum2Tag (SEQ ID NO. 33) | GTPIVIMVDEAKPSLP |
| Rum3Tag (SEQ ID NO. 34) | GNPLIVMIDEAEQKEI |
| Rum4Tag (SEQ ID NO. 35) | AGGIIVMKDNTTKVSI |
| Rum5Tag (SEQ ID NO. 36) | GNPIVTMIDDATLVKI |
| Rum6Tag (SEQ ID NO. 37) | GNSTITMVDDTTKVHI |
| Rum7Tag (SEQ ID NO. 38) | GTPLIVMVDDTTKVEI |
| BacTag (SEQ ID NO. 39) | NEKVTGQFEIVKVDANDKTK |
| Bac2Tag (SEQ ID NO. 40) | SKSLGQFEIVKVDAQDKTK |
| Bac3Tag (SEQ ID NO. 41) | LGQFEIVKVDSQDKTK |
| Bac4Tag (SEQ ID NO. 42) | VTGQFEIVKVDAEDKTR |
| Bac5Tag (SEQ ID NO. 43) | EKVMGQFEIMKVDANDKTK |

Key
*may refer to a stop codon at the end of the sequence. Preferably, said sequence should end .DSATHI,
in which case * is nothing.

Variants, derivatives and modifications of the binding pairs may be made by any suitable means. Variants, derivatives and functionally operative modifications may involve amino acid additions, substitutions, alterations or deletions that retain the same function in relation to the ability to form an isopeptide bond with the relevant binding partner.

The peptide pairs may be defined by reference to a sequence. The sequence may be identical for the sequence listed for the peptide partner. The peptide partner may have a sequence which has at least 60%, 70%, 80%, 85%, 90%, 95% or 99% sequence identity to the listed sequence. Variants and derivatives of the peptide partner may comprise an amino acid sequence that is at least 90% or 95% similar to the listed sequence. Homologues of these entities may therefore have at least 60% homology thereto, such as at least 65%, such as at least 70%, such as at least 75%, such as at least 80%, such as at least 85%, such as at least 90%, such as at least 91%, such as at least 92%, such as at least 93%, such as at least 94%, such as at least 95%, such as at least 96%, such as at least 97%, such as at least 98%, such as at least 99% homology thereto.

For some of the binding pairs, mediation by a third entity such as an enzyme is required. For example, SnoopLigase may be used to meditate the bond formation between SnoopTagJr and DogTag. Thus, the pairing may require the assistance of an enzyme such as a ligase.

### Antigen

An antigen as used herein refers to any molecule that is capable of inducing immune responses. An antigen can be a, allergenic antigen, viral antigen, bacterial antigen, parasitic antigen or fungal antigen. “Antigen” as used herein includes peptides and epitopes, variants and derivatives thereof. A tumour antigen includes tumour-specific antigen, tumour-associated antigen and neoantigens, newly formed antigens by cancerous cells. “Tumour-specific antigen” refers to antigens that are only found on tumour cells. “Tumour-associated antigen” refers to antigens presented by both tumour and normal cells. “Neoantigen” refers to newly formed antigens by tumour cells. “Antigen” as used herein includes peptides and epitopes, variants and derivatives thereof.

### B Cell Epitope

As used herein a B cell epitope is the part of an antigen that is recognised by the immune system specifically by antibodies and B cells.

### T Cell Epitope

As used herein a T cell epitope is the part of the antigen that is recognised by T cells.

Tumour-associated antigens that can be used as the antigen to decorate the vector or be included as the transgene include, but are not limited to adipophilin, AIM-2, ALDH1A1, alpha-actinin-4, alpha-fetoprotein (“AFP”), ARTC1, B-RAF, BAGE-1, BCLX (L), BCR-ABL fusion protein b3a2, beta-catenin, BING-4, CA-125, CALCA, carcinoembryonic antigen (CEA), CAGE 1 to 8, CASP-5, CASP-8, CD274, CD45, Cdc27, CDK12, CDK4, CDKN2A, CEA, CLPP, COA-1, CPSF, CSNK1A1, CTAG1, CTAG2, cyclin DI, Cyclin-A1, dek-can fusion protein, DKK1, EFTUD2, Elongation factor 2, ENAH (hMena), Ep-CAM, EpCAM, EphA3, epithelial tumor antigen (ETA), ETV6-AML1 fusion protein, EZH2, ErbB receptors, E6, E7, FGF5, FLT3-ITD, FN1, G250/MN/CAIX, GAGE-1,2,8, GAGE-3,4,5,6,7, GAS7, glypican-3, GnTV, gp100/Pmel 17, GPNMB, HAUS3, Hepsin, HER-2/neu, HERV-K-MEL, HLA-A11, HLA-A2, HLA-DOB, hsp70-2, HPV E2, HPV E6, HPV E7 antigen, IDO1, IGF2B3, IL13Ralpha2, Intestinal carboxyl esterase, K-ras, Kallikrein 4, KIF20A, KK-LC-1, KKLC1, KM-HN-1, KMHN1 also known as CCDCl10, LAGE-1, LDLR-fucosyltransferase fusion protein, Lengsin, M-CSF, MAGE-A1, MAGE-A 10, MAGE-A12, MAGE-A2, MAGE-A3, MAGE-A4, MAGE-A6, MAGE-A9, MAGE-C1, MAGE-C2, malic enzyme, mammaglobin-A, MART2, MATN, MC1R, MCSP, mdm-2, ME1, Melan-A/MART-1, Meloe, Midkine, MMP-2, MMP-7, MUC1, MUC5AC, mucin, MUM-1, MUM-2, MUM-3, Myosin, Myosin class I, N-raw, NA88-A, neo-PAP, NFYC, NY-BR-1, NY-ESO-I/LAGE-2, OA1, OGT, OS-9, P polypeptide, p53, PAP, PAX5, PBF, pml-RARalpha fusion protein, polymorphic epithelial mucin (PEM), PPP1R3B, PRAME, PRDX5, PSA, PSMA, PTPRK, RAB 38/N Y-MEL-1, RAGE-1, RBAF600, RGS5, RhoC, R F43, RU2AS, SAGE, secernin 1, SIRT2, SNRPD1, SOX10, Spl7, SPA17, SSX-2, SSX-4, STEAP1, survivin, SYT-SSX1 or -SSX2 fusion protein, TAG-1, TAG-2, Telomerase, TGF-betaRII, TPBG, TRAG-3, Triosephosphate isomerase, TRP-I/gp75, TRP-2, TRP2-INT2, tyrosinase (TYR), VEGF, WT1, XAGE-Ib/GAGED2a,

Those skilled in the art of identifying tumour-associated antigens will appreciate that new antigens, including neoantigens, are continually identified, and as such this list is not exhaustive.

Viral antigens that can be used to decorate the vector or be used as the transgene include, but are not limited to antigens of the following viruses or class of viruses; Human Papilloma Viruses (HPV) eh L1 or L2 capsid protein, Human Immunodeficiency virus (HIV) eg gp120/gp160, Herpes Simplex Virus (HSV2/HSV1) eg gD and gl, Influenza virus (types A, B and C), Polio virus, Respiratory Syncitial Virus (RSV) e.g. F antigen included those stabilised in the pre-fusion form, Rhinoviruses, Rotaviruses, Hepatitis A virus, Norwalk Virus Group, Enteroviruses, Astroviruses, Measles virus, Parainfluenza virus, Mumps virus, Varicella-Zoster virus e.g. gE, Human Cytomegalovirus (HCMV) e.g. gB, Epstein-Barr virus, Adenoviruses, Rubella virus, Human T-cell Lymphoma type I virus (HTLV-I), Hepatitis B virus (HBV) e.g. hepatitis B surface antigen, Hepatitis C virus (HCV), Hepatitis D virus, Poxviruses, Marburg virus and Ebola virus, SARS-Cov-2 e.g. spike or S protein.

Similarly bacterial antigens include, but are not limited to antigens of the following bacteria: Mycobacterium tuberculosis, Chlamydia, Neisseria gonorrhoeae, Shigella, Salmonella, Vibrio cholerae, Treponema pallidum, Pseudomonas, Bordetella pertussis, Brucella, Francisella tularensis, Helicobacter pylori, Leptospira interrogans, Legionella pneumophila, Yersinia pestis, Streptococcus (types A and B), Pneumococcus, Meningococcus, Haemophilus influenzae (type b), Toxoplasma gondii, Campylobacter, Moraxella catarrhalis, Klebsiella granulomatis and Actinomyces.

Likewise fungal antigens include, but are not limited to antigens of the following fungal pathogens: Candida and Aspergillus, Cryptococcus, Histoplasma and Pneumocystis.

Parasitic antigens include, but are not limited to antigens of the following parasitic pathogens: Taenia, Flukes, Roundworms, Plasmodium, Amoeba, Giardia, Cryptosporidium, Schistosoma, Trichomonas, Trypanosoma and Trichinella.

### Pharmaceutical Composition and Use

The compositions of the invention may be incorporated into a vaccine or therapeutic composition. Suitably, a vaccine or immunogenic composition will comprise particles of the invention in an immunogenic dose.

A pharmaceutical composition may comprise a particle or composition in accordance with the invention provided with a pharmaceutically acceptable carrier. Suitable carriers are well known to those skilled in the art. In one embodiment a pharmaceutical composition comprises a buffer, excipient or carrier. Suitably a pharmaceutical composition may comprise suitable excipients and formulations to maintain stability of the composition. Suitably the formulation may comprise an adjuvant. In one embodiment, the formulation may comprise AddaVax™ or a similar squalene-based oil-in-water nano-emulsion with a formulation similar to MF59®. Other suitable adjuvants include liposome-based adjuvants such as Matrix M and AS01. Other suitable adjuvants include aluminium-based formulations such as Alhydrogel®. In one embodiment the formulation may comprise EDTA, for example at a concentration of 5 mM. Suitable excipients or formulations may depend on the properties of the particle or immunogenic composition; for example, the choice of expression system may affect the stability, glycosylation or folding of the proteins of the composition, which may in turn affect the optimal formulation of the composition. Methods of determination of a suitable excipient, formulation or adjuvant will be known to those skilled in the art.

### Vaccine

A vaccine is a preparation that comprises a fragment or entire entity against which it is possible to raise an immune response. It is an entity such as a protein, peptide, lipoprotein, glycoprotein, polysaccharide or fragments thereof that are capable of inducing an immune response. For example, the vaccine may comprise micro-organisms or a part thereof capable of inducing an immune response against said micro-organism. A vaccine comprising an immunogenic adenoviral vector in accordance with the invention can be used against any pathogen for which the antigen displayed or encoded by the transgene for the induction of an immune response against the antigen

Such vaccine compositions (or other immunogenic) are formulated in a suitable delivery vehicle. Generally, doses for the immunogenic compositions are within the ranges defined for therapeutic compositions. Optionally, a vaccine composition of the invention may be formulated to contain other components, including, for example, adjuvants, stabilizers, pH adjusters, preservatives and the like. Such components are well known to those skilled in the art of vaccines. Examples of suitable adjuvants include, without limitation, liposomes, alum, monophosphoryl lipid A, saponins, such as Qs21, and any biologically active factor, such as a cytokine, an interleukin, a chemokine and optimally combinations thereof. It will be appreciated that whist adjuvants may be used, generally, the adenoviral vaccines of the invention are not adjuvanted.

The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, locally and/or using aerosol, injection, infusion, continuous infusion, localized perfusion bathing target cells directly or via a catheter and/or lavage. Typically the vaccines of the invention are administered intramuscularly.

The vaccine may be used to treat or prevent infection with any one of the disease-causing pathogens hereinbefore described or be used to treat tumours.

### Ad-DogTag

The “Ad-DogTag” viral vector comprises the insertion of DogTag into surface loops of the hexon capsid protein enabling display of up to ˜720 ligands/virion. Coupling of an antigen to hexon-DogTag has been achieved by the present inventors using SnoopTagJr-tagged antigens (using SnoopLigase as a catalyst) or directly via DogCatcher linked antigens. Previous technologies have only been capable of inserting small immunogenic T cell or B cell epitopes with a length of <100 amino acids into adenovirus hexon loops. The present invention demonstrates the coupling of peptides of 10-60 kDa to hexon, which has not previously been possible to achieve. This represents a big step forwards in the development of vaccines based upon adenovirus in particular.

### Ad-SpyCatcher

The “Ad-SpyCatcher” viral vector comprises the fusion of SpyCatcher onto the C-terminus of adenovirus minor capsid protein pIX. The recent invention was successful in modifying the pIX minor capsid protein without loss of viral infectivity.

### Ad-SnoopCatcher

The “Ad-SnoopCatcher” viral vector comprises the fusion of SnoopCatcher onto the C-terminus of adenovirus minor capsid protein pIX. The inventors have had success in modifying the pIX minor capsid protein without loss of viral infectivity.

### Ad-DogCatcher

The “Ad-DogCatcher” viral vector comprises the fusion of DogCatcher onto the C-terminus of adenovirus minor capsid protein pIX. The inventors have had success in modifying the pIX minor capsid protein without loss of viral infectivity.

### Ad-SnoopTagJr

The “Ad-SnoopTagJr” viral vector comprises the fusion of SnoopTagJr onto the C-terminus of adenovirus minor capsid protein pIX. The inventors have had success in modifying the pIX minor capsid protein without loss of viral infectivity.

### Vaccination

In the Examples detailed below we describe a flexible, rapid, and targeted system of spontaneous covalent decoration of recombinant adenovirus particles with a variety of ligands and under mild conditions. In particular, using the DogTag/DogCatcher reactive pair, we targeted the hexon protein for capsid display to maximize ligand density since hexon is the most abundant component of the adenovirus capsid. Several previous studies have achieved display of short immunogenic epitopes by direct genetic insertion of sequences into hexon HVR loops, but these have typically been limited to <50 residues since larger insertions disrupt protein stability and prevent successful rescue of recombinant vectors. Other studies have performed chemical modification of the viral capsid to attach larger ligands, including targeted insertion of reactive disulphide groups on cysteine residues, but these approaches can require challenging reaction and storage conditions to achieve conjugation and retain infectivity of modified vectors.

Using our approach, we have covalently coupled ligands as large as the RBD of SARS CoV-2 Spike (˜26 kDa) to hexon through genetic fusion to DogCatcher and co-incubation of the recombinant fusion protein with Ad-DogTag, achieving extensive coverage of the viral capsid in each case. Importantly, and despite a high proportion of hexon monomers conjugated to ligand, decorated vectors retained infectivity in vitro. Transduction of 293A cells by Ad5 vectors has been shown to be mediated by interactions between the adenovirus fiber protein and the coxsackie and adenovirus receptor (CAR), these interactions are unlikely to be masked by hexon conjugation due to the sizes of the ligands tested, as reflected by the cryoEM data (FIG. 7).

Decoration of the Ad capsid surface with both DogCatcher-SARS CoV-2 Spike-RBD and Influenza HA-RBD reduced the potency of anti-Ad neutralizing antibodies in vitro. Capsid shielding against mAb9C12 was effective, likely due to coverage of the capsid surface with the RBD ligands. Indeed, cryoEM data indicated that all hexon trimers had at least one copy of SARS CoV-2 Spike-RBD attached, with ligand density covering much of the hexon trimer surface (FIG. 6). Shielding from neutralization by polyclonal anti-Ad serum was also achieved. Studies have shown that serum post vaccination with Ad vectors typically contains antibodies against other viral proteins in addition to hexon (including fiber) which are less likely to be masked by hexon ligation. Our platform has the potential to shield the Ad virion from other potentially undesirable capsid interactors. For instance, display of DogCatcher-SARS CoV-2 Spike-RBD blocked hFX mediated transduction of SKOV-3 cells in vitro, presumably by inhibiting interaction between hFX and hexon. Shielding this interaction in vivo would be advantageous since hFX has been shown to enhance Ad5 transduction of hepatocytes leading to liver sequestration and hepatotoxicity after systemic administration. To date, Ad capsid shielding solutions have predominantly involved coating virions with polymers such as polyethylene glycol (PEG) or N-(2-hydroxypropyl)methacrylamide (HPMA). Although effective capsid shields, these polymer coatings have tended to impair vector transduction.

Capsid decoration with DogCatcher-SARS CoV-2 Spike-RBD significantly enhanced boosting of humoral immunity against SARS CoV-2 using Ad vaccine vectors. In homologous prime-boost regimens, decorating an Ad vector encoding Spike with DogCatcher-SARS CoV-2 Spike-RBD increased median anti-RBD antibody responses >10 fold compared to the undecorated Ad. Strikingly, a vector encoding an irrelevant antigen (GFP) but with SARS CoV-2 Spike-RBD displayed on the Ad capsid also elicited significantly higher anti-RBD antibody responses compared to the undecorated Ad encoding Spike. Key to the enhanced humoral immunity achieved with our platform was increased boostability of antibody responses against capsid displayed SARS CoV-2 Spike-RBD. Fold-increase in anti-RBD antibody titers between prime and boost was ˜10-fold higher with RBD capsid display compared to the undecorated Ad. A similar observation was made after a heterologous regimen using SARS CoV-2 Spike-RBD decorated Ads to boost immunity following immunization with a conventional undecorated Ad encoding Spike (FIG. 6). In both experiments, boosting of humoral immunity using homologous prime-boost with undecorated Ad encoding Spike was modest (˜2-5-fold increase in anti-RBD ELISA, FIGS. 3D and 4B), and these data are comparable to previous reports using other Ad vectors encoding SARS CoV-2 Spike in mice. In trials of CoVID-19 vaccines in humans, boosting of cellular and humoral immunity using homologous Ad prime-boost regimens has also been modest, particularly when compared to heterologous regimens with mRNA vaccines or homologous mRNA regimens. Anti-vector immunity generated after a prime immunization may inhibit the ability to boost immune responses to encoded antigens using the same vector; anti-Ad neutralizing antibodies prevent vector transduction and subsequent transgene antigen expression required for antigen-specific immunity. In contrast, recombinant protein antigens displayed in particulate form on nanoparticles or VLPs (in adjuvant formulations) have been shown to generate robust humoral immunity, with highly efficient boosting in homologous prime-boost regimens. Previous studies have suggested that particulate antigens generate potent antibody responses through a variety of mechanisms including B cell receptor crosslinking and prolonged retention in draining lymph nodes. It seems likely that responses against capsid decorated SARS CoV-2 Spike-RBD are induced by similar mechanisms, particularly since Ad particles are of optimum size for lymph node entry and retention, though future studies will be required to elucidate these mechanisms further.

The term “Boostability” used herein refers to the ability of an adaptive immune response (either antibody or T cell) to be boosted upon repeat administration of a vaccine. Conventional adenovirus vaccines typically exhibit modest boostability of immune responses against transgene encoded antigens upon repeat administration due to anti-vector immunity generated after the first dose. Particulate protein vaccines (such as VLPs or decorated Ads as in the present invention) on the other hand, show effective boosting of antibody responses upon repeat administration.

While capsid antigen decoration induced potent humoral immunity, encoding of antigen sequences in the vector genome was required to generate potent cellular immunity. Importantly, capsid decoration did not impair T cell responses to encoded antigens, and in fact a heterologous prime-boost regimen using capsid decorated vectors at boost improved cellular immunity compared to a homologous regimen (FIGS. 6C and 6D). The latter observation suggests that our capsid shielding technology applied at boost may have circumvented anti-vector immunity generated after a priming immunization. In support of this hypothesis, median SARS CoV-2 Spike specific IFNγ ELISPOT responses were comparable between homologous prime-boost regimens using decorated and undecorated vectors encoding Spike in both experiments but were ˜2 fold higher in the heterologous regimen (FIG. 6C).

Our capsid display platform offers a significant improvement over conventional Ad vaccine technology for induction of both cellular and humoral immunity. Taken together, the data presented here support a concept for optimising concomitant humoral and cellular immunogenicity, whereby antigenic targets for antibody induction are displayed on the capsid surface, and potent T cell epitopes are encoded in the vector genome. Such a concept would enable conserved components of target pathogens that induce potent T cell responses (such as nucleocapsid or ORF1ab of SARS CoV-2) to be encoded in the vector genome, while targets of neutralizing humoral immunity such as Spike-RBD (which are often more heterogeneous in sequence identity) can be displayed on the capsid surface and even exchanged for different versions in response to emergence of new variants if necessary. Finally, the ability of this platform to induce both robust cellular and humoral immunity and to enhance efficacy of multi-shot regimens could be advantageous for applications beyond prophylactic vaccines including therapeutic vaccines against chronic viral pathogens and cancer. Methods of rapid and customizable covalent decoration of Ad capsids could also be utilized for development of personalized therapies.

### EXAMPLES

Certain aspects and embodiments of the invention will now be illustrated by way of example and with reference to the figures described herein.

All materials, methods and examples described herein detail specific examples that fall within the scope of the present invention and support the understanding thereof. In addition, this disclosure is illustrative only, so is not limited to any particular methods and experimental conditions described. As such methods and conditions may vary and it is not intended to be limiting in any way. Although alternative methods and materials similar or equivalent to those described herein can be used in the practice or testing of the present disclosure, suitable methods and materials are described below.

### Example 1:—Materials and Methods

### Generating a Bacterial Artificial Chromosome (BAC)-Derived Replication-Defective Molecular Clone of Ad5 Expressing GFP

Plasmid pAd-PL-DEST, an E1/E3-deleted (and therefore replication-defective) molecular clone of Ad5, was obtained from Invitrogen. An expression construct, consisting of an immediate early cytomegalovirus promoter (CMVp) containing tetracycline operator sequences driving expression of enhanced green fluorescent protein (EGFP), was cloned into shuttle vector pENTR4 (Invitrogen). The CMVp EGFP expression construct was then inserted into the Ad5 E1 locus using Invitrogen Gateway site-specific recombination (LR clonase) technology. BAC sequences from pBELOBAC11 (NEB) were amplified using forward (5′-TTAATTAAcgtcgaccaattctcatg) and reverse (5′-TTAATTAAgtcgacagcgacacacttg) primers to introduce Pac/sites at either end of the BAC cassette. The entire Ad5(GFP) genome sequence was subsequently cloned into the BAC with Pacl, to generate pBAC-Ad5(GFP).

Genetic Modification of pBAC-Ad5(GFP) to Insert DogTag Protein Superglue Technology into Viral Capsid Proteins Using BAC GalK Recombineering

SW102, an E. coli strain required for GalK recombineering, was obtained from the National Cancer Institute, National Institutes of Health, USA. Modified from DH10B, SW102 cells contain A-Red-encoded recombination genes (exo, bet, gam) under the control of a temperature-sensitive repressor with a deleted galactokinase (GalK) gene (which is necessary for bacterial growth using galactose as the sole carbon source). The GalK recombineering system enables the GalK gene to be used for both positive and negative selection, and GalK recombineering was performed exactly as described in Warming et al, 2005 [Warming S, Costantino N, Court DL, Jenkins NA, Copeland NG. Simple and highly efficient BAC recombineering using galK selection. Nucleic Acids Res. 2005;33(4):e36]. An insertion site was created in hexon HVR5 loop (as described in FIG. 2A) by insertion of the GalK gene at this specific locus by recombineering followed by positive selection for the presence of GalK. Insertion of the DogTag sequence was performed by recombineering to replace GalK, and subsequent selection against the presence of the GalK gene using 2-deoxygalactose. The resulting vector was termed Ad5-HVR5-DogTag.

Generation of pBAC-Ad5 HVR5 DogTag Expressing SARS CoV-2 Spike (1-1208)

A pAd-DEST (Invitrogen Gateway) version of Ad5-HVR5-DogTag was generated through BP Clonase mediated site specific recombination between pBAC-Ad5(GFP)-HVR5-DogTag described previously and pDONR221 (with the chloramphenicol resistance gene in pDONR221 replaced with an ampicillin resistance gene). BP clonase mediated recombination between attB sites on pBAC-Ad5(GFP)-HVR5-DogTag and attP sites on pDONR replaced the GFP gene with ccdB and generated attR sites flanking the E1 locus to generate pBAC Ad5-HVR5-DogTag DEST. The SARS CoV-2 Spike protein, residues 1-1208 (Wuhan strain, including stabilising mutations K986P and V987P and mutation of the furin cleavage site 682-GSAS-685, codon optimised for mammalian expression) was cloned into shuttle vector pENTR4 (Invitrogen) downstream of CMVp, and then inserted into the E1 locus of pBAC Ad5-HVR5-DogTag DEST using LR Clonase (Invitrogen).

### Transfection, Growth and Rescue of Recombinant Adenoviruses Incorporating Protein Superglue Technology

BAC DNA from recombinant adenovirus molecular clones was linearised with Pacl to release left and right viral inverted terminal repeats (ITRs). Linearised DNA was transfected into E1-complementing Human Embryonic Kidney (HEK) 293 cell lines (either 293A cells (Invitrogen) for Ad5 vectors expressing GFP or 293TREX cells (Invitrogen) for Ad5 vectors expressing SARS CoV-2 Spike) in 25 cm2 flasks (T25) using Lipofectamine 2000 reagent (Invitrogen). After cytopathic effect (CPE) was observed, the cells were harvested, subjected to three cycles of freeze-thaw, and the virus amplified further in HEK293A cells. Upon infection of 10×150 cm2 flasks (T150), virus was harvested from infected cells after 48 hours and purified by CsCl gradient ultracentrifugation according to standard protocols. Purified virus was dialysed against sucrose storage buffer (10 mM Tris-HCl, 7.5% w/v sucrose, pH 7.8) and stored at −80° C.

### Estimation of Viral Particle Count for Purified Viral Vector Preparations

The number of adenovirus particles in a purified preparation can be estimated by measuring viral DNA content by spectrophotometric absorption at 260 nm as described by Maizel et al, 1968. [J. Maizel, D. White, M. Scharff, The polypeptides of adenovirus: I. Evidence for multiple protein components in the virion and a comparison of types 2, 7A, and 12. Virology, Volume 36, Issue 1, September 1968, Pages 115-125]. Briefly, samples were diluted 1:10 in virus storage buffer containing 1% w/v sodium dodecyl sulphate (SDS) to release viral DNA from capsids and absorbance at 260 nm was measured using a spectrophotometer. An absorbance of 1.00 (AU, 1 cm pathlength) at 260 nm corresponds to 1.1×10¹² viral particles/mL.

### Infectious Titration of Recombinant Adenoviruses in HEK293A Cells

Infectious titre of vector preparations was assessed by single cell infectivity assay on HEK293A cells or 293TREX cells. For vectors expressing EGFP, infected HEK293A cells were visualised and enumerated directly by fluorescent microscopy. An alternative assay was used for vectors without a fluorescent marker, by immunostaining for expression of the hexon capsid protein in either 293A or 293TREX cells.

Tenfold serial dilutions of virus in complete media (Dulbecco's Modified Eagles Medium, plus 1×GlutaMAX and 10% v/v foetal bovine serum) were performed in sterile 96-well deep well plates. Two or three serial dilutions were performed per virus, and 50 μL of each dilution (10³ to 10¹⁰ dilution) was added per well of a 96-well plate containing adherent HEK293 cells at 80-90% confluency. For hexon immunostaining, 96-well plates pre coated with poly-L-Lysine were used to improve cell adhesion to the plate surface. Plates were incubated for 48 hours at 37° C., 5% CO₂. For titration of EGFP-expressing vectors, 48 hours post infection, single GFP-positive cells were enumerated by fluorescence microscopy, and an infectious titre was calculated in infectious units (ifu) per mL. For hexon immunostaining, media was aspirated from the cell monolayer and cells were fixed with ice-cold methanol. Plates were then washed three times in Dulbecco's phosphate buffered saline (PBS 1×, Gibco) before blocking for an hour with 3% w/v low-fat milk (Marvel). Detecting mouse monoclonal anti-hexon antibody (B025/AD51, Thermo-Fisher) was added at 1:1000 dilution in 1% w/v milk in PBS and incubated for an hour at 25° C. After incubation of the primary antibody, cells were washed a further three times with 1% w/v milk in PBS prior to addition of a secondary goat anti-mouse alkaline phosphatase (ALP) conjugated antibody (STAR117A, BioRad) at 1:1000 dilution in 1% w/v milk in PBS. After a further hour incubation, plates were washed five times in PBS prior to development. To develop, 100 μL of freshly prepared SIGMAfast BCIP/NBT solution (Sigma) was added to each well and plates incubated at 25° C. until the appearance of dark stained foci, representing single infected cells. P:I ratios for CsCl-purified vector preparations were calculated by dividing the estimated no. of viral particles per mL with the no. of infectious units (ifu) per mL.

### Production of Protein and Peptide Ligands

DNA sequences for expression of polyhistidine-tagged recombinant DogCatcher with 18 copies of the NANP repeat sequence from the Plasmodium falciparum circumsporozoite protein fused to the C-terminus (DogCatcher-NANP18) were cloned into expression plasmid pET45(+) (EMD Millipore) for protein production in BL21(DE3) E. coli. (NEB). Recombinant proteins were purified using affinity Ni-NTA resin (Qiagen) according to a previously published protocol [SnoopLigase Catalyzes Peptide-Peptide Locking and Enables Solid-Phase Conjugate Isolation. Buldun CM, Jean JX, Bedford MR, Howarth M. J Am Chem Soc. 2018 Feb. 28; 140(8):3008-3018. doi: 10.1021/jacs.7b13237], dialysed into PBS, and stored at −80° C.

DNA sequences for expression of DogCatcher fused to SARS CoV-2 Spike receptor binding domain (Wuhan strain, residues 319-532) (DogCatcher-S-RBD), SnoopTagJr fused to Spike receptor binding domain (S-RBD-SnJr) and DogCatcher fused to the receptor binding domain of haemagglutinin (HA) from Influenza A (H1 A/California/04/2009, residues 55-266 based on H3 numbering) (DogCatcher-HA-RBD) were cloned into mammalian protein expression plasmid pcDNA3.4. DogCatcher was fused at the N-terminus of S-RBD and HA-RBD, and SnoopTagJr was fused at the C-terminus of S-RBD. To facilitate secretion, the IgK leader sequence METDTLLLWVLLLWVPGSTGD (SEQ ID NO. 46) was introduced at the N-terminus of each of these fusion proteins. A C-terminal C-tag (EPEA) was fused to each construct to enable affinity purification. RBD-SnoopTagJr was expressed in suspension Expi293F cells, and DogCatcher-S-RBD and DogCatcher-HA-RBD were expressed in suspension ExpiCHO-S cells. Protein was harvested from culture supernatant, affinity purified using C-tag affinity resin (Thermo Fisher) using an AKTA chromatography system (GE), and dialysed into tris-buffered saline (TBS) pH 7.4.

### Coupling Reactions

For in vitro assays, coupling reactions between DogCatcher-fused protein ligands and DogTag inserted into the Ad5 capsid (at hexon HVR5) were performed by co-incubation of spontaneously reacting components in a total volume of 20 μL, with individual components at concentrations described in the figure legends. Reactions were incubated for 16 h at 4° C.

Ligand-decorated vector batches for the NANP vaccine studies were prepared by co-incubating 1.9E±12 viral particles of Ad5(GFP)-HVR5-DogTag with 35 μM DogCatcher-NANP18 for 16 hours at 4° C. To remove excess ligand, coupled vectors were dialysed into sucrose storage buffer using SpectraPor dialysis cassettes (300 kDa MWCO). Dialysis reduced excess ligand by over 10-fold as measured on a coomassie-stained SDS-PAGE gel. Ligand coverage by SDS-PAGE was >90% hexon.

Ligand-decorated vector batches for SARS CoV-2 vaccine studies and electron microscopy were prepared by co-incubating 9E+11 viral particles Ad5-HVR5-DogTag encoding either GFP or SARS CoV2 Spike with 6 μM DogCatcher-S-RBD for 16 hours at 4° C. To remove excess ligand, coupled vectors were dialysed into sucrose storage buffer using SpectraPor dialysis cassettes (300 kDa MWCO). Dialysis reduced excess ligand by at least 20-fold as measured on a coomassie-stained SDS-PAGE gel.

For all decorated vaccine vectors, residual excess ligand post dialysis was factored into effective antigen dose calculations. Ligand-decorated vectors were stored at −80° C., endotoxin tested, and infectious titration of stored batches was repeated prior to immunisation.

### Assessment of Coupling Efficiency by SDS-PAGE

Coupling reactions were performed as described above and stopped by addition of SDS loading buffer (BioRad, 31.5 mM Tris-HCl, pH 6.8, 10% glycerol, 1% SDS, 0.005% Bromophenol Blue, 300 mM DTT). Samples were boiled at 95° C. for 5 min and loaded on SDS-PAGE (NuPAGE 4-12% Bis-Tris, Invitrogen) gels. Coupling efficiency of ligand coupling (for example DogCatcher-fused ligands) to the adenoviral capsid (for example Ad5-HVR-DogTag, expressed as % total hexon protein coupled) was assessed by direct gel shift assays. Proteins were resolved by SDS-PAGE (200V, 55 min) and visualized by Coomassie staining [16 h staining with Quick Coomassie (Generon), destained with water]. Coupling efficiency was calculated by comparing band intensities of unconjugated hexon-Tag in ligand decorated samples to undecorated (control) samples using Image J:

$$Efficiency ( \% hexon coupled ) = \frac{hexon - Tag ( control ) - hexon - Tag ( decorated )}{hexon - Tag ( control )} \times 100$$

### Anti-Vector Antibody Neutralisation Assay

For assessment of vector neutralisation by potent neutralising mouse monoclonal antibody (mAb) 9C12 (Developmental Studies Hybridoma Bank, University of Iowa), Ad5 vectors expressing GFP were incubated with serially diluted mAb 9C12 antibody at a 1:1 ratio in complete media for 1 hour at 37° C. The vector-antibody mix was then added to an 80% confluent monolayer of HEK293A cells in a 96-well plate format (cells were infected at a multiplicity of infection of 200 ifu/cell). Cells were incubated with the vector-antibody mix for 2 hours at 37° C. 5% CO₂, before the mix was replaced with fresh media and the plates returned to 37° C. 5% CO₂ for a further 24 h. After 24 h, GFP expression within HEK293A cells was used as a readout of vector infectivity; bulk fluorescence was measured on a fluorimeter (Tecan) using an excitation wavelength of 395 nm and emission wavelength of 509 nm.

For assessment of vector neutralisation by Ad5-positive serum, serum samples were obtained by immunising mice with 1E+8 ifu of an Ad5 vector expressing ovalbumin (vector had an unmodified hexon). Serum was harvested two-weeks post immunization, stored at −80° C., and then serially diluted for the neutralisation assay (two-fold dilutions were prepared from 1:4 to 1:512 in complete media, to give a final range of 1:8 to 1:1024 on cell monolayers). Diluted serum was incubated with Ad5(GFP) vectors, the mix incubated on HEK293 cells and bulk GFP fluorescence read 24 h later exactly as described above.

### Assessment of Coagulation Factor X-Mediated Vector Transduction of SKOV3 Cells

SKOV3 cells (human ovary adenocarcinoma) were cultured in McCoy's 5a media with 2 mM Glutamine and 15% v/v foetal bovine serum (complete McCoy's). For the assay, GFP-expressing Ad vectors were serially diluted (1:10 to 1:107) in serum free media. Human coagulation Factor X (FX) (Haematologic Technologies) was added to diluted vectors at a final concentration of 8 μg/mL. Ad/FX mixes were added to monolayers of SKOV3 cells in 96-well plates, and incubated with cells for 2.5 h at 37° C. and 5% CO₂. After 2h, vector-FX mixtures were replaced with complete McCoy's media, and plates incubated at 37° C., 5% CO₂ for a further 48h. Infectivity was assessed after 48h by enumeration of GFP-positive foci as described above.

### Mouse Immunisations

All mouse procedures were performed in accordance with the terms of the UK Animals (Scientific Procedures) Act Project Licence (PA7D20B85) and approved by the Oxford University Ethical Review Body. Female Balb/c mice (6 weeks of age, Charles River), housed in specific-pathogen free environments, were immunised intramuscularly by injection of 50 μl of vaccine formulated in endotoxin-free PBS (Gibco) into both hind limbs of each animal (100 μL total). Doses of adenoviral vectors and recombinant proteins administered are described in figure legends. Protein vaccines were administered in combination with adjuvants as described. Alhydrogel (Invivogen) was administered at a 1:9 v/v ratio of adjuvant to antigen. AddaVax (Invivogen) was administered at a 1:1 v/v ratio of adjuvant to antigen. Endotoxin dose was <3 EU per mouse in all studies. Experiments were performed at Biomedical Services, University of Oxford.

### Ex-vivo IFN-gamma ELISPOT

Overnight spleen ex vivo interferon-gamma (IFN-γ) ELISpot was performed according to standard protocols as described previously [Larsen K C, Spencer A J, Goodman AL, Gilchrist A, Furze J, Rollier C S, Kiss-Toth E, Gilbert SC, Bregu M, Soilleux E J, Hill A V, Wyllie D H, Expression of tak1 and tram induces synergistic pro-inflammatory signalling and adjuvants DNA vaccines. Vaccine. 2009 Sep. 18; 27(41):5589-98]. To measure antigen specific responses, cells were re-stimulated with peptides for 18-20 hours. To measure T cell responses to GFP, peptide CD8+ T cell epitope EGFP ₂₀₀₋₂₀₈ was added at a final concentration of 5 μg/mL. To measure T cell responses to SARS CoV-2 Spike receptor binding domain (S-RBD), cells were stimulated with a peptide pool of 15-mer peptides with an 11 amino acid overlap spanning the length of the S-RBD region (PODTC2 319-541, PepMix™ SARS CoV-2 S-RBD, JPT peptide technologies). Pooled peptides were added at a final concentration of 0.5 μg/mL/peptide. To measure T cell responses to SARS CoV-2 Spike protein, cells were stimulated with two peptide pools of 15-mer peptides with an 11 amino acid overlap spanning the entire length of the Spike protein (PepMix™ SARS CoV-2 Spike Glycoprotein, JPT peptide technologies). Pooled peptides were added at a final concentration of 0.5 μg/mL/peptide. Spot forming cells (SFC) were measured using an automated ELISpot reader system (AID).

### Igg ELISA

IgG endpoint ELISA was performed as described previously [S. Biswas, M. D. Dicks, C. A. Long, E. J. Remarque, L. Siani, S. Colloca, et al. Transgene optimization, immunogenicity and in vitro efficacy of viral vectored vaccines expressing two alleles of Plasmodium falciparum AMA1 PLOS ONE, 6 (2011), p. e20977]. Plates were coated with either recombinant GFP protein (Millipore) at 1 μg/mL to measure GFP specific responses, recombinant DogCatcher-NANP18 protein a concentration of 2 μg/ml to measure DogCatcher-NANP18 specific responses, or recombinant SARS CoV-2 S-RBD protein (Wuhan strain, Spike residues 319-532) expressed in CHO-S cells fused to SnoopTagJr and C-Tag at a concentration of 2 μg/mL to measure SARS CoV-2 S-RBD specific responses. To generate endpoint titers, sera from mice immunised with Ad5 vectors expressing an irrelevant antigen (GFP for DogCatcher-NANP18 and SARS CoV-2 specific assays, DogCatcher-NANP18 for GFP specific assays) was used as a negative control.

### CryoEM Data Collection and Image Processing

CryoEM analysis was performed by Nanolmaging Services, San Diego, USA. A 3 μl drop of sample Ad5-Tag (Control) and sample Ad5-Tag:Catcher-RBD were applied to a 1/2 Cu-mesh C-flat grid that had been plasma-cleaned for 10s using a 25% O2/75% Ar mixture in a Solarus 950 Plasma Cleaner (Gatan). Grids were manually plunge frozen in liquid ethane. Data collection was carried out using a Thermo Fisher Scientific Glacios Cryo Transmission Electron Microscope operated at 200 kV and equipped with a Falcon 3 direct electron detector. Automated data-collection was performed with Leginon software (57) at a nominal magnification of 28,000×, corresponding to a pixel size of 5.19 Å. A total of 845 and 617 movies were recorded for samples Ad5-Tag and Ad5-Tag:Catcher-RBD, respectively, using a nominal defocus range of −2.4 to −5.6 μm. Exposures were fractionated into 19 frames with an exposure rate of 2.6 e-/pixel/s and total exposure of 10 e-/Å2.

For both samples, motion correction and CTF estimation were performed using cryoSPARC3.1. Using the Gaussian blob picker in cryoSPARC3.1, a total of 28,371 particles were picked for sample Ad5-Tag:Catcher-RBD and 91,920 particles were picked for sample Ad5-Tag. These particles were extracted from the cryoTEM images and subjected to reference-free 2D classification in the cryoSPARC3.1. The best 28,307 particles for sample Ad5-Tag:Catcher-RBD and all particles picked for sample Ad5-Tag were used for the subsequent 3D reconstruction. An initial model was generated ab initio from all selected particles for sample Ad5-Tag: Catcher-RBD and used during one round of homogenous refinement with icosahedral symmetry imposed in cryoSPARC3.1. The final 3D reconstruction of Ad5-Tag: Catcher-RBD was lowpass filtered to 30 Å and used as a starting volume for the 3D reconstruction of sample Ad5-Tag during one round of homogenous refinement with icosahedral symmetry imposed in cryoSPARC3.1. The resolution of the final 3D reconstructions was determined by the Fourier shell correlation (FSC) between two independent half maps, which was 10.5 Å at FSC=0.143 for both samples. Maps are visualized using Chimera.

### Example 2: Concept of Modular Capsid Decoration of Adenovirus

The hexon protein is the major component of the adenovirus capsid (each virion comprising 720 copies assembled into 240 trimers) and is therefore an ideal target for modular capsid display. The inventors utilized the DogTag/DogCatcher reactive pair to achieve spontaneous covalent coupling of DogCatcher fused ligands onto the surface of adenovirus virions.

Referring to FIG. 1, a viral capsid is shown formed from the hexon protein (hexagon) with the DogTag shown as attached to the hexon protein and displayed on the outside of the viral capsid.

To enable covalent capsid decoration, DogTag (23 amino acids) was genetically inserted into a hexon hypervariable surface loop. Here, the DogTag is shown to be flanked by flexible glycine-serine linker sequences. As herein described other construct designs with and without linkers and/or a linker inserted upstream and/or downstream of the Tag protein are envisaged.

Referring to FIG. 2 shows the design of the DogCatcher-SARS CoV-2 S-RBD (C-RBD) ligand, with fusion of residues 319-532 from SARS CoV-2 Spike (the receptor binding domain) to the C-terminus of Dogcatcher. An N-terminal Igk leader sequence facilitates secretion of the protein upon expression in CHO cells. DogCatcher-SARS CoV-2-RBD protein was designed for display on the surface of the adenovirus capsid. The DogCatcher reacts with the DogTag decorating the surface of the viral capsid to achieve spontaneous covalent coupling of DogCatcher ligands onto the surface of adenovirus virons. A flexible linker is shown between the catcher (DogCatcher) and the antigen (e.g., SARS CoV-2 S-RBD protein).

### Example 3—Antibody Responses Against Catcher-NANP Generated Through Adenovirus Capsid Surface Display

The circumsporozoite protein (CSP) of Plasmodium falciparum (Pf) has been extensively studied as a malaria vaccine candidate antigen. The protein contains a highly immunogenic repetitive region, primarily consisting of repeats of the sequence NANP; vaccine induced antibodies against this NANP repeat region have been shown to protect against malaria infection. Previously, (WO2021/084282) a recombinant fusion protein consisting of DogCatcher fused at the N-terminus of a protein sequence consisting of 18 consecutive NANP repeats (DogCatcher-NANP18) was generated by expression in E.Coli. Previous data has shown that this fusion protein can be coupled to Ad5 with DogTag inserted into the HVR5 hexon loop (Ad-DogTag). Subsequent display of DogCatcher-NANP18 on the adenovirus capsid surface is able to shield the viral capsid from vector neutralising antibodies in vitro.

An immunogenicity study in mice was conducted to assess antibody titers against the DogCatcher-NANP18 protein when displayed on the adenovirus capsid surface, compared to immunization with an adenovirus vector encoding the DogCatcher-NANP18 construct, or DogCatcher-NANP18 recombinant protein in adjuvant formulations (FIG. 3A). Two weeks after a single vaccine dose, IgG endpoint titers against DogCatcher-NANP18 were significantly higher after administration of an adenovirus vector displaying DogCatcher-NANP18 on the capsid sequence (Ad(GFP)-T:C-NANP18), than after administration of a comparable dose of a vector encoding DogCatcher-NANP18 (Ad(C-NANP18)) or recombinant protein in adjuvant (FIG. 3B). Importantly, display of the DogCatcher-NANP18 ligand on the capsid surface did not impair immune responses against the encoded GFP transgene; GFP CD8+ T cell responses (FIG. 3C) and GFP IgG antibody responses (FIG. 3D) were equivalent between a vector with (Ad(GFP)-T:C-NANP18) and without (Ad(GFP)-T) a capsid ligand.

### Example 4—Display of Catcher-SARS CoV-2 S-RBD on the Adenovirus Capsid Provides a Shield from Anti-Vector Neutralising Antibodies

A fusion protein consisting of DogCatcher fused to the N-terminus of the receptor binding domain of SARS CoV-2 Spike protein (S-RBD) was generated and expressed in mammalian Chinese hamster ovary (CHO) cells. Ad-DogTag encoding a GFP transgene (1E+10 viral particles) was incubated with DogCatcher-S-RBD (3.5 μM) overnight (16h) at 4° C. Samples were run on SDS-PAGE and proteins visualised by Coomassie staining as shown in FIG. 4A. Coupling efficiency (expressed as % total hexon protein coupled) was calculated to be 64%.

Immediately after coupling, a vector infectivity assay was performed on the samples described in FIG. 4A. Samples were serially diluted, applied to monolayers of HEK293A cells in a 96-well plate, and incubated for 48 h at 37° C. with 5% CO₂. Infectious titres were calculated by enumeration of GFP fluorescent foci by fluorescence microscopy. Infectious titres were calculated as no. of infectious units (ifu) per ml. Data indicated that vector infectivity was retained after ligand coupling (FIG. 4B).

Coupled vector was incubated with serially diluted virus-neutralising mouse monoclonal anti-hexon antibody mAb 9C12 (Developmental Studies Hybridoma Bank, University of Iowa) for 1 h at 37° C., before the vector-antibody mix was added to an 80% confluent monolayer of HEK293A cells in a 96-well plate format (cells were infected at a multiplicity of infection of 200 ifu/cell). Cells were incubated with the vector-antibody mix for 2 h at 37° C. with 5% CO₂, before the mix was replaced with fresh media and the plates returned to 37° C. with 5% CO₂ for a further 24 h. After 24 h, GFP expression within HEK293A cells was employed as a readout of infectivity—bulk fluorescence was measured on a fluorimeter using an excitation of 395 nm and emission of 509 nm. The data, shown in FIG. 4C, demonstrate that Ad-DogTag displaying DogCatcher-S-RBD (Ad-T:C-RBD) shows little reduction in infectivity in the presence of increasing concentrations of mAb 9C12 up to 50 ng/ml, while Ad-DogTag without a capsid shield (Ad-T) is effectively neutralised by mAb 9C12.

In FIG. 4D, a neutralisation assay with Ad5-positive mouse serum (instead of mAb 9C12) was performed on coupled Ad-T:C-RBD. To generate Ad5-positive mouse serum, mice were immunised intramuscularly with 1E+8 ifu Ad5(Ovalbumin) (WT hexon) vector (Ad5 E1 and E3 deleted vector with an unmodified hexon expressing ovalbumin instead of GFP under the control of a constitutive CMV promoter encoded at E1 locus) and serum was harvested two-weeks post-immunisation. The data show that neutralizing anti-serum exhibits reduced potency against Ad-T:C-RBD compared to Ad-T.

In FIG. 4E, an assay was performed to test human Factor X mediated transduction of SKOV-3 cells. Human coagulation factor X (hFX) has previously been shown to bind directly to the Ad5 hexon protein and mediates hepatocyte transduction via heparan sulphate proteoglycans (HSPGs) by Ad5 vectors in vivo, particularly after intravascular delivery. In an in vitro model of this interaction, capsid decoration with DogCatcher-S-RBD completely abrogated hFX mediated Ad5 transduction of SKOV-3 cells while Ad5 without a capsid shield exhibited a 6.6-fold increase in SKOV-3 infectivity upon co-incubation with hFX (FIG. 4E).

### Example 5—High-Titer Antibody Responses Generated Against SARS CoV-2 S-RBD when Displayed on the Adenovirus Capsid Surface

An immunogenicity study in mice was conducted to assess antibody titers against the SARS CoV-2 S-RBD protein when displayed on the adenovirus capsid surface, compared to an adenovirus vector with the SARS CoV-2 Spike protein (residues 1-1208) encoded in the vector genome.

Mice were immunised in a homologous prime-boost regimen (on Day 0 and Day 21) with either Ad-DogTag encoding Spike 1-1208 (Ad(Spike)-T, Group 1), the same vector as in Group 1 but with DogCatcher-S-RBD coupled to the viral capsid (Ad(Spike)-T:C-RBD, Group 2), Ad-DogTag encoding a GFP transgene with DogCatcher-S-RBD coupled to the viral capsid (Ad(GFP)-T:C-RBD, Group 3) or with recombinant DogCatcher-S-RBD protein (C-RBD) in either alhydrogel (Group 4) or Addavax (Group 5) adjuvants (FIG. 5A).

Serum antibody titers (IgG endpoint ELISA) against SARS CoV-2 S-RBD pre-boost (Day 20, FIG. 5B) and two weeks post-boost (Day 35, FIG. 5C) are shown. Post-boost, antibody titers were higher after immunisation with vectors displaying SARS CoV-2 S-RBD on the capsid surface (Groups 2 and 3) compared to the vector only encoding the Spike protein (Group 1) (FIG. 3C). Indeed, antibody titers after immunisation with Ad(GFP)-T:C-RBD (Group 3) were comparable to Ad(Spike)-T:C-RBD (Group 2) despite the former vector lacking an encoded Spike antigen. FIG. 3D shows the fold change in ELISA titer between Day 20 and Day35, and indicates that display of SARS CoV-2 RBD on the capsid surface (Group 2 and 3) increased the boostability of the antibody response compared with the Spike encoding vector without capsid display (Group 1).

Importantly, display of the DogCatcher-S-RBD ligand on the capsid surface did not impair T cell responses against encoded SARS CoV-2 Spike antigens. Spleen IFNγ T cell ELISPOT responses against a SARS CoV-2 S-RBD peptide pool (FIG. 5E) and a SARS CoV-2 S (full-length) peptide pool (FIG. 5F) showed that T cell immunogenicity with Ad(Spike)-T and Ad(Spike)-T:C-RBD was comparable. T cell responses against SARS CoV-2 S-RBD were significantly higher in magnitude in Groups 1 and 2 compared with Group 3, indicating that encoding an antigen is preferable to capsid display for inducing T cell immunogenicity.

Taken together, the data support a concept for optimising concomitant humoral and cellular immunogenicity using adenovirus vectors, whereby antigenic targets for antibody induction are displayed on the capsid surface, and potent T cell epitopes are encoded in the vector genome.

### Example 6: Applying a Capsid Shield to Booster Adenovirus Vaccines Improves Both Humoral and Cellular Immunogenicity

Given the ability of capsid display technology to elicit potent humoral immunity against SARS CoV-2, we sought to determine whether RBD capsid decoration could efficiently boost immunity in animals that had already received a first dose of a conventional Ad vector encoding Spike. Homologous Ad(Spike)-T prime-boost was compared to a heterologous regimen in which an RBD capsid shield (Ad(Spike)-T:C-RBD) was applied to the second vaccine dose (FIG. 6A). A 5.3-fold increase in median anti-RBD IgG titers was observed between prime and boost immunizations in the homologous regimen, but a >10-fold higher (63.1-fold) increase in titer was observed after the heterologous regimen (FIG. 6B). In addition, spleen IFNγ ELISPOT responses against full-length SARS CoV-2 Spike were also higher in the heterologous regimen compared to the homologous regimen (FIG. 6C). A similar observation was observed in IFNγ ELISPOT assays using a peptide pool spanning only residues 633-1273 of Spike (hence not including the RBD region), indicating that increased cellular immunity to the encoded Spike transgene after heterologous prime-boost occurred independently from the contribution of the RBD capsid ligand (FIG. 6D).

### Example 7: CryoEM Analysis of Adenovirus Particles Displaying SARS CoV-2 Spike-RBD

To further investigate the nature of capsid decoration of Ad particles with SARS CoV-2 Spike-RBD, cryoEM analysis of decorated (Ad-Tag:Catcher-RBD) and undecorated (Ad-Tag) particles was performed (FIG. 7). A 3D density map of decorated particles clearly indicated additional density extending outward from hexon trimers compared to the undecorated particles (FIG. 7A). Additional density protruding from hexon trimers could also be observed from 2D class averages of Ad-Tag:Catcher-RBD particles compared to Ad-Tag particles, extending particle diameter from 93 nm to 98 nm (FIG. 7B). Surface protrusions (representing hexon trimers) on Ad-Tag were of uniform density, while Ad-Tag:Catcher-RBD showed two distinct regions, the outermost region representing coupled RBD being less dense than hexon trimers likely due to flexibility of the attached ligand. Analysis of 3D density maps revealed attachment of either one (type I, FIG. 7C) or two (type II. FIG. 7D) RBD ligands per hexon trimer. Type II decoration was particularly abundant on hexon trimers located adjacent to penton subunits at capsid vertices. These trimers are elevated with respect to the rest of the virion surface (see FIG. 7A, darker regions of Ad-Tag map; note that the fiber protein is not shown) and therefore steric hindrance may be lower, enabling multiple ligand occupancy. Additional density representing attached RBD is clear for both type I and type II arrangement compared to undecorated Ad-Tag; FIG. 7E shows fitting of an RBD structure into these maps (with the RBD N-terminus proximal to hexon HVR5, DogCatcher is not shown). This cryoEM data indicated that all hexon trimers had at least one copy of RBD attached, with ligand density covering much of the hexon trimer surface.

### Example 8—Display of Catcher-HA-RBD on the Adenovirus Capsid Provides a Shield from a Potent Anti-Capsid Neutralising Antibody

A fusion protein consisting of DogCatcher fused to the N-terminus of the receptor binding domain of Influenza A haemagglutinin receptor binding domain (H1 A/California/04/2009 strain, HA-RBD) was generated and expressed in mammalian Chinese hamster ovary (CHO) cells. Ad-DogTag encoding a GFP transgene (5E+9 viral particles) was incubated with DogCatcher-HA-RBD (1.75 μM) overnight (16h) at 4° C. Samples were run on SDS-PAGE and proteins visualised by Coomassie staining as shown in FIG. 8A. Coupling efficiency was calculated to be 44%.

Immediately after coupling, a vector infectivity assay was performed on the samples described in FIG. 4A. Samples were serially diluted, applied to monolayers of HEK293A cells in a 96-well plate, and incubated for 48 h at 37° C. with 5% CO₂. Infectious titres were calculated by enumeration of GFP fluorescent foci by fluorescence microscopy. Infectious titres were calculated as no. of infectious units (ifu) per ml. Data indicated that vector infectivity was retained after ligand coupling (FIG. 8B).

Coupled vector was incubated with serially diluted virus-neutralising mouse monoclonal anti-hexon antibody mAb 9C12 for 1 h at 37° C., before the vector-antibody mix was added to an 80% confluent monolayer of HEK293A cells in a 96-well plate format (cells were infected at a multiplicity of infection of 200 ifu/cell). Cells were incubated with the vector-antibody mix for 2 h at 37° C. with 5% CO₂, before the mix was replaced with fresh media and the plates returned to 37° C. with 5% CO₂ for a further 24 h. After 24 h, GFP expression within HEK293A cells was employed as a readout of infectivity—bulk fluorescence was measured on a fluorimeter using an excitation of 395 nm and emission of 509 nm. The data, shown in FIG. 7C, demonstrate that Ad-DogTag displaying DogCatcher-HA-RBD (Ad-T:C-RBD) shows only a modest reduction in infectivity in the presence of increasing concentrations of mAb 9C12 up to 100 ng/ml, while Ad-DogTag without a capsid shield (Ad-T) is effectively neutralised by mAb 9C12 at concentrations >10 ng/ml.

Taken together, the data presented in FIG. 4 and FIG. 8 demonstrate that receptor binding domains from diverse pathogenic viruses can be displayed on the capsid surface of adenovirus vectors. Decoration of the adenovirus capsid with these RBD domains as Catcher fusion proteins can also shield the capsid to effectively block binding of vector neutralising antibodies and/or other undesirable capsid interactions as a generalisable concept.

1. An adenoviral vector encoding a heterologous transgene encoding an antigen and wherein the vector has at least one modified capsid protein, said modification comprises the inclusion of a first peptide partner covalently bonded to a second peptide partner, wherein the second partner is attached to an antigen, characterised in that the antigen encoded by the transgene has at least one T cell epitope and the antigen attached to the second partner has at least one B cell epitope.

2. An adenoviral vector as claimed in claim 1 encoding a heterologous transgene encoding an antigen and wherein the vector has at least one modified capsid protein, said modification comprises the inclusion of a first peptide partner covalently bonded to a second peptide partner, wherein the second partner is attached to an antigen, characterised in that the antigen encoded by the transgene and the antigen attached to the second partner share at least one epitope.

3. An adenoviral vector as claimed in claim 1 or 2 wherein the antigen encoded by the transgene and the antigen attached to the second partner are derived from the same pathogen.

4. A vector as claimed in claims 1 to 3 wherein the amino acid sequence of antigen encoded by the transgene and the amino acid sequence of the antigen attached to the second partner are 40, 50, 60, 70, 80, 90, 95, 97,99, 100% identical.

5. A vector as claimed in any preceding claim wherein the antigen encoded by the transgene or the antigen attached to the second partner is an antigen selected from the group viral, bacterial, parasitic, or fungal antigen.

6. A vector as claimed in any preceding wherein the antigen attached to the second partner is the receptor binding domain of the antigen.

7. An adenoviral vector as claimed in any of claims 1 to 3, or claim 5 or 6, wherein the transgene encodes an HCMV pentamer and the antigen attached to the second partner comprises HCMV gB or a fragment thereof.

8. An adenoviral vector as claimed in any of claims 1-3, or claim 5 or 6, wherein the transgene encodes Nucleoprotein from Influenza virus and the antigen attached to the second partner is an influenza heamagglutinin or fragment thereof.

9. An adenoviral vector of claim 8 wherein the heamagglutinin fragment comprises the receptor binding domain (RBD).

10. An adenoviral vector as claimed in any one of claims 1 to 6 wherein the transgene encoded antigen and the antigen attached to the second partner is an S antigen or fragment thereof from SARS CoV-2.

11. A vector as claimed in claim 10 wherein the S antigen fragment is the receptor binding domain.

12. A vector as claimed in any of claims 1 to 11, wherein the modified capsid protein is a hexon protein.

13. A vector as claimed in claims 1 to 11 wherein the modified capsid protein is a hexon protein and in modified a HVR loop.

14. A vector as claimed in claims 1 to 11 wherein the modified capsid protein is a pIX protein

15. A vector as claimed in any of claims 1 to 14 wherein the modification to the capsid protein is the insertion or fusion of the first peptide partner.

16. A vector as claimed in claimed in any preceding claim wherein the first peptide is covalently bonded to the second peptide and the covalent bond is an isopeptide bond.

17. A vector as claimed in any preceding claim wherein the first and second peptide partners are selected from the group of first and second pairs:
SpyCatcher and SpyTag;
SnoopCatcher and SnoopTag/SnoopTagJr
DogCatcher and DogTag
SnoopTagJr and SnoopCatcher
DogTag and SnoopTag/SnoopTagJr using SnoopLigase
SpyTag and SpyCatcher
provided that when the modified capsid protein is a hexon protein the first partner is not SpyCatcher.

18. A vector as claimed in any preceding claim wherein the antigen attached to the second peptide is over 15, Kda, 20Kda, 30Kda, 40Kda, 60Kda, 70Kda, 80Kda,90Kda, or 100 Kda in size.

19. A vaccine comprising the adenoviral vector of any preceding claim.

20. A vector as claimed in any of claims 1 to 18 and a vaccine as claimed in claim 19 for use in medicine.

21. Use of a vector of any of claims 1 to 18 in the manufacture of a medicament for the treatment or of prophylaxis disease.

22. A method of treating a patient in need thereof, comprising administering a safe and effective amount of a vaccine of claim 19 or a vector of any one of claims 1 to 18.

23. A method of producing a vector of claims 1 to 18 comprising:
i. Introducing a nucleic acid which encodes a first peptide partner into the nucleic acid encoding a capsid protein of an adenovirus
ii. Introducing a transgene encoding an antigen into the adenoviral genome
iii. Infecting a cell with the adenovirus and collecting the progeny
iv. Attaching a second peptide partner attached to an antigen to the first peptide partner, wherein the antigen encoded by the transgene has at least one T cell epitope, and the antigen attached to the second peptide partner has at least one B cell epitope.

24. A method of manufacturing a vaccine of claim 19, comprising admixing the vector of any of claims 1 to 18 with a pharmaceutically acceptable excipient.


SEQUENCE LISTING (46 sequences)

SEQ ID NO: 1 - protein, 113 aa, Artificial Sequence
  SpyCatcher
VDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 2 - protein, 92 aa, Artificial Sequence
  SpyCatcher N1
DSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 3 - protein, 113 aa, Artificial Sequence
  SpyCatcher002
VTTLSGLSGEQGPSGDMTTEEDSATHIKFSKRDEDGRELAGATMELRDSSGKTISTWISDGHVKDFYLYPGKYTFVETAAPDGYEVATAITFTVNEQGQVTVNGEATKGDAHT

SEQ ID NO: 4 - protein, 113 aa, Artificial Sequence
  Spycatcher003
VTTLSGLSGEQGPSGDMTTEEDSATHIKFSKRDEDGRELAGATMELRDSSGKTISTWISDGHVKDFYLYPGKYTFVETAAPDGYEVATPIEFTVNEDGQVTVDGEATEGDAHT

SEQ ID NO: 5 - protein, 83 aa, Artificial Sequence
  SpyCatcher N1 C2
DSATHIKFSKRDEDGKELAGATMELRDSSGKTISTWISDGQVKDFYLYPGKYTFVETAAPDGYEVATAITFTVNEQGQVTVNG

SEQ ID NO: 6 - protein, 112 aa, Artificial Sequence
  SnoopCatcher
KPLRGAVFSLQKQHPDYPDIYGAIDQNGTYQNVRTGEDGKLTFKNLSDGKYRLFENSEPAGYKPVQNKPIVAFQIVNGEVRDVTSIVPQDIPATYEFTNGKHYITNEPIPPK

SEQ ID NO: 7 - protein, 98 aa, Artificial Sequence
  Spyligase
DYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 8 - protein, 98 aa, Artificial Sequence
  SdyCatcher
LSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 9 - protein, 104 aa, Artificial Sequence
  RrgACatcher
KLGDIEFIKVNKNDKKPLRGAVFSLQKQHPDYPDIYGAIDQNGTYQNVRTGEDGKLTFKNLSDGKYRLFENSEPAGYKPVQNKPIVAFQIVNGEVRDVTSIVPQ

SEQ ID NO: 10 - protein, 104 aa, Artificial Sequence
  DogCatcher V1
KLGEIEFIKVDKTDKKPLRGAVFSLQKQHPDYPDIYGAIDQNGTYQDVRTGEDGKLTFTNLSDGKYRLIENSEPPGYKPVQNKPIVSFRIVDGEVRDVTSIVPQ

SEQ ID NO: 11 - protein, 104 aa, Artificial Sequence
  DogCatcher V2
KLGEIEFIKVDKTDKKPLRGAVFSLQKQHPDYPDIYGAIDQNGTYQDVRTGEDGKLTFTNLSDGKYRLFENSEPPGYKPVQNKPIVAFQIVDGEVRDVTSIVPQ

SEQ ID NO: 12 - protein, 91 aa, Artificial Sequence
  PsCsCatcher
EQDVVFSKVNVAGEEIAGAKIQLKDAQGQVVHSWTSKAGQSETVKLKAGTYTFHEASAPTGYLAVTDITFEVDVQGKVTVKDANGNGVKAD

SEQ ID NO: 13 - protein, 282 aa, Artificial Sequence
  PilinC
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 14 - protein, 113 aa, Artificial Sequence
  QueenCatcher
IDTMSGLSGETGQSGNTTIEEDSTTHVKFSKRDSNGKELAGAMIELRNLSGQTIQSWVSDGTVKDFYLMPGTYQFVETAAPEGYELAAPITFTVNEQGQVTVNGKATKGDAHI

SEQ ID NO: 15 - protein, 112 aa, Artificial Sequence
  Kat I
DTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVQIQGKATRGDVPI

SEQ ID NO: 16 - protein, 113 aa, Artificial Sequence
  Mooncake
IDTMSGLSGETGQSGNTTIEEDSTTHVKFSKRDSNGKELAGAMIELRNLSGQTIQSWVSDGTVKDFYLMPGTYQFVETAAPEGYELAAPITFTVQDNGEVIIQGRLTRGDVHI

SEQ ID NO: 17 - protein, 13 aa, Artificial Sequence
  SpyTag
AHIVMVDAYKPTK

SEQ ID NO: 18 - protein, 14 aa, Artificial Sequence
  SpyTag002
VPTIVMVDAYKRYK

SEQ ID NO: 19 - protein, 16 aa, Artificial Sequence
  SpyTag003
RGVPHIVMVDAYKRYK

SEQ ID NO: 20 - protein, 12 aa, Artificial Sequence
  SnoopTag
KLGDIEFIKVNK

SEQ ID NO: 21 - protein, 12 aa, Artificial Sequence
  SnoopTag Jr
KLGSIEFIKVNK

SEQ ID NO: 22 - protein, 23 aa, Artificial Sequence
  DogTag
DIPATYEFTDGKHYITNEPIPPK

SEQ ID NO: 23 - protein, 13 aa, Artificial Sequence
  SdyTag
DPIVMIDNDKPIT

SEQ ID NO: 24 - protein, 13 aa, Artificial Sequence
  PsCsTag
GNKLTVTDQAAPS

SEQ ID NO: 25 - protein, 19 aa, Artificial Sequence
  RrgATag
DIPATYEFTNDKHYITNEP

SEQ ID NO: 26 - protein, 16 aa, Artificial Sequence
  IsopepTag
TDKDMTITFTNKKDAE

SEQ ID NO: 27 - protein, 60 aa, Artificial Sequence
  RumTag
AGCGAAAACGGCAACCCGCTGATTGTGATGGTGGATGATACCACCAAAGTGAAAATTAGC

SEQ ID NO: 28 - protein, 16 aa, Artificial Sequence
  Clib9
RGVPTIVMVDCYKRYK

SEQ ID NO: 29 - protein, 15 aa, Artificial Sequence
  RumTrunkTag
GNPLIVMVDDTTKVK

SEQ ID NO: 30 - protein, 15 aa, Artificial Sequence
  RumTrunk D9N Tag
GNPLIVMVNDTTKVK

SEQ ID NO: 31 - protein, 22 aa, Artificial Sequence
  PhoTag
LVTGTAHIVMVDNYKPIVETGD

SEQ ID NO: 32 - protein, 15 aa, Artificial Sequence
  EntTag
NTIVMVDKLKEVPPT

SEQ ID NO: 33 - protein, 16 aa, Artificial Sequence
  Rum2Tag
GTPIVIMVDEAKPSLP

SEQ ID NO: 34 - protein, 16 aa, Artificial Sequence
  Rum3Tag
GNPLIVMIDEAEQKEI

SEQ ID NO: 35 - protein, 16 aa, Artificial Sequence
  Rum4Tag
AGGIIVMKDNTTKVSI

SEQ ID NO: 36 - protein, 16 aa, Artificial Sequence
  Rum5Tag
GNPIVTMIDDATLVKI

SEQ ID NO: 37 - protein, 16 aa, Artificial Sequence
  Rum6Tag
GNSTITMVDDTTKVHI

SEQ ID NO: 38 - protein, 16 aa, Artificial Sequence
  Rum7Tag
GTPLIVMVDDTTKVEI

SEQ ID NO: 39 - protein, 20 aa, Artificial Sequence
  BacTag
NEKVTGQFEIVKVDANDKTK

SEQ ID NO: 40 - protein, 19 aa, Artificial Sequence
  Bac2Tag
SKSLGQFEIVKVDAQDKTK

SEQ ID NO: 41 - protein, 16 aa, Artificial Sequence
  Bac3Tag
LGQFEIVKVDSQDKTK

SEQ ID NO: 42 - protein, 17 aa, Artificial Sequence
  Bac4Tag
VTGQFEIVKVDAEDKTR

SEQ ID NO: 43 - protein, 19 aa, Artificial Sequence
  Bac5Tag
EKVMGQFEIMKVDANDKTK

SEQ ID NO: 44 - DNA, 26 bp, Artificial Sequence
  BAC sequences pBELOBAC11 forward primer
ttaattaacgtcgaccaattctcatg

SEQ ID NO: 45 - DNA, 27 bp, Artificial Sequence
  BAC sequences pBELOBAC11 reverse prim
ttaattaagtcgacagcgacacacttg

SEQ ID NO: 46 - protein, 21 aa, Artificial Sequence
  IgK leader sequence
METDTLLLWVLLLWVPGSTGD